(19) United States
(12) Patent Application Publication
(10) Pub. No.: US 2025/0002608 A1
(43) Pub. Date: Jan. 2, 2025

(54) TREATMENT OF PAIN

(71) Applicant: UTI Limited Partnership, Calgary (CA)

(72) Inventors: Christophe ALTIER, Calgary (CA); Gerald Werner ZAMPONI, Calgary (CA)

(21) Appl. No.: 18/709,750

(22) PCT Filed: Nov. 14, 2022
(86) PCT No.: PCT/CA2022/051678

§ 371 Date: May 13, 2024

Related U.S. Application Data
(60) Provisional application No. 63/279,287, filed on Nov. 15, 2021.

(51) Int. Cl. C07K 16/40; A61K 31/7088; A61P 29/00; C07K 16/28

(52) U.S. Cl. CPC C07K 16/40; A61K 31/7088; A61P 29/00; C07K 16/28

(57) ABSTRACT

In one aspect, there is a provided a method of treating pain in a subject in need thereof comprising administering to the subject an anaplastic lymphoma kinase (ALK) inhibitor. In one aspect, there is a provided a method of treating pain in a subject in need thereof comprising administering to the subject an ALKAL2 inhibitor.

### CROSS REFERENCE TO RELATED APPLICATION

This application claims priority to United States Provisional Patent Application U.S. 63/279,287, filed Nov. 15, 2021, the entire contents of which is hereby incorporated by reference.

### FIELD

The present disclosure relates generally to the treatment of pain.

### BACKGROUND

Sensitization of nociceptive neurons can lead to persistent pain in response to inflammation or injury. Identifying the mechanisms of peripheral sensitization has been key to defining the maladaptive long-lasting changes in nociceptive circuits which ultimately precipitate the transition to chronic pain (1-3). During inflammation, this sensitization process occurs particularly in thinly myelinated A6 and unmyelinated C-fibers that express the Transient Receptor Potential Vanilloid 1 (TRPV1) channel (2-5) and transduce inflammatory stimuli. Despite characterization of a large number of inflammatory mediators, their receptors and downstream signaling pathways, very few of these targets have led to efficacious treatments for pain relief (6, 7).

### SUMMARY

In one aspect there is provided a method of treating pain in a subject in need thereof comprising administering to the subject an anaplastic lymphoma kinase (ALK) inhibitor.

In one example, the ALK inhibitor is a small molecule, an antibody, a polynucleotide, or a pharmaceutical composition thereof.

In one example, the ALK inhibitor is Crizotinib, Alectinib, Brigatinib, Lorlatinib, or TPX-0131.

In one example, wherein the antibody is a monoclonal antibody, preferably mAb30, mAb49, or anti-Human ALK/CD246 Monoclonal Antibody.

In one example, wherein the polynucleotide is an antisense polynucleotide to target human ALK hASO-1: 5′-GAAGCAGAGCGCACACAAAA (SEQ ID NO: 25), hASO-2: 5′-TCCTCATCCATGGGCTCAGA (SEQ ID NO: 26), hASO-3: 5′-CGCTGAGGTTGAACTGGAGT (SEQ ID NO: 27).

In one example, the pain is inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer, including “break-through pain” and pain associated with terminal cancer, peripheral neuropathy and post-herpetic neuralgia.

In one example, the subject is a cat, a dog, a cow, a horse, a pig, a sheep, a goat, a mouse, a rabbit, a rat, a guinea pig, a non-human mammal, a non-human primate, a rodent, a bird, a reptile, an amphibian, or a fish.

In one example, the subject is a human subject.

In one aspect there is provided a method of treating pain in a subject in need thereof comprising administering to the subject an ALKAL2 inhibitor.

In one example, the ALKAL2 inhibitor is a small molecule, an antibody, a polynucleotide, or a pharmaceutical composition thereof.

In one example, the polynucleotide is an antisense polynucleotide. Human specific ASO: Design #1: hASO-1 DNA Sequence: 5′-CAAGTACACTGATTTATCGA (SEQ ID NO: 4). Design #2: hASO-2 DNA Sequence: 5′-GCACTACACGTCAAATGTGG (SEQ ID NO: 5). Design #3: hASO-3 DNA Sequence: 5′-ACAATAGCTGGAATACTATT (SEQ ID NO: 6).

In one example, the antisense polynucleotide is Design #1: ASO-1 DNA Sequence: 5′-AAGTGCTTGCTGCACTTCGG (SEQ ID NO: 1), Design #2: ASO-2 DNA Sequence: 5′-GATGGTGCAGTCTCTCGTGT (SEQ ID NO: 2), or Design #3: ASO-3 DNA Sequence: 5′-TGGTGTGTCGCTCCTTTGCA (SEQ ID NO: 3).

In one example, the pain is inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer, including “break-through pain” and pain associated with terminal cancer, peripheral neuropathy and post-herpetic neuralgia.

In one example, the subject is a cat, a dog, a cow, a horse, a pig, a sheep, a goat, a mouse, a rabbit, a rat, a guinea pig, a non-human mammal, a non-human primate, a rodent, a bird, a reptile, an amphibian, or a fish.

In one example, the subject is a human subject.

In one example, administration is topical, intradermal, intramuscular, intraperitoneal, intravenous, subcutaneous, intranasal, epidural, or oral routes.

### BRIEF DESCRIPTION OF THE FIGURES

Embodiments of the present disclosure will now be described, by way of example only, with reference to the attached Figures.

FIG. 1. Characterization of the TRPV1-pHluorin knock in mouse. (A) PHluorin was inserted into the turret region of the TRPV1 channel, between residue H614 and K615 using the CRISPR-Cas9 technology (31, 32). (B) Representative confocal images showing GFP expression in dorsal root ganglion (DRG), Nodose Ganglion (NG) and spinal cords of WT (left) and TRPV1-pHluorin (right) mouse. (C) Co-immunostaining of GFP and TRPV1 in DRG (left panel); co-immunostaining of GFP with markers of peptidergic (CGRP) or non-peptidergic (IB4) neurons in the spinal dorsal horn (right panel). (D) Western blot of DRG lysates from WT and TRPV1-pHluorin mice using an anti-GFP antibody, note the specific band at ˜125 kDa only in the transgenic mice (left panel). Immunoprecipitation of TRPV1-pHluorin from DRG lysates using GFP-trap. Membranes were then probed with an anti-TRPV1 antibody (right panel). Note the absence of band in WT animals. (E) Representative whole cell current-clamp recordings of the capsaicin-evoked AP discharge in DRG neurons from WT and TRPV1-pHluorin mice. (F) Dose-response curve evoked by capsaicin, measured by calcium imaging on DRG neurons from WT (circles) (EC50=0.91±0.12 μM, n=34) or TRPV1-pHluorin mice (squares) (EC50=1.06±0.09 μM, n=28) at room temperature (22° C.).

FIG. 2. CFA inflammation induces ALKAL2 upregulation in TRPV1 neurons. (A) Experimental approach used to conduct the microarray analysis from TRPV1 neurons, 72 hours after intraplantar CFA. (B) FACS isolation of TRPV1-pHluorin neurons. Representative FACS plot of GFP+ population in WT (top) and TRPV1-pHluorin (bottom) mice. (C) Dot plot showing the average score obtained by comparing the top 200 genes of the Affymetrix dataset with the nociceptor populations identified in the Renthal's dataset (see Method). (D) Scatter plot representation of genes regulated in CFA conditions. Genes that pass a threshold of log 2 fold change in differential expression analysis are colored in green when they are downregulated and red when they are upregulated. All genes are listed in Table 1. (E) qRT-PCR assessment of Alkal2 upregulation in the DRG ipsilateral to the CFA injection, compared to the contralateral side and naïve control. (F) Representative Western blot of ALKAL2 in the DRG ipsilateral to the CFA injection, compared to the contralateral side. Below, quantification of ALKAL2 protein level from the western blot experiments. Each dot represents a sample collected from a different animal (n=6). Results indicate the mean±SEM. Statistical analysis was performed using one-way ANOVA (E) followed by Bonferroni post hoc test (*p<0.05) and t-test (F) (**p<0.01).

FIG. 3. Characterization of ALKAL2 expression. (A) ALKAL2 expression in neuronal and non-neuronal cells of the CNS and PNS, from a mouse single-cell RNA-sequencing data set (Linnarsson's lab; http://mousebrain.org/genesearch.html) (60). (B) Representative western blot showing the level of ALKAL2 protein in the CNS and PNS. Note the greater signal intensity in DRGs. (C) Representative confocal images of co-immunostaining for ALKAL2 and TRPV1, 1B4, GFRα3 and NF200 in DRG neurons. Scale bar: 50 μm. Note the abundance of ALKAL2 in peptidergic (TRPV1, GFRα3) DRG neurons. (D) Whisker plot summarizing the results in (C), 77.64±2.95% of TRPV1, 77.64±3.06% of GFRα3, 41.65±7.08% of 1B4 and 36.46±2.64% of NF200 positive neurons, express ALKAL2 (each symbol represents a DRG section from 4 individual animal). (E) ALKAL2+ fibers in the sciatic nerve are unmyelinated C fibers that do not express NF200. (F) Representative RNAscope image showing expression of ALKAL2 (light blue) in human DRG neurons co-expressing Nav1.8 (pink). On the right, bar graph summarizing the results (each symbol represents an individual patient). Results indicate the mean±SEM.

FIG. 4. ALKAL2 induces sprouting and hyperexcitability of small DRG neurons. (A) Schematic illustrating the co-culture system used to chronically expose DRG neurons to ALKAL2. HEK cells were plated into the upper chamber of a transwell and then transfected with ALKAL2 plasmid for 16 h. Cells were washed and DRG neurons were plated in the lower chamber of the transwell for another 16 h of co-culture and then immunostained for Tuj1. (B) ALKAL2 induces a significant increase of total neurites in small (<20 μm) DRG neurons (10±2.5 vs 18.6±1.2 in control and ALKAL2 plasmid, respectively); number of branch points per neuron (5.4±0.8 vs 8.9±0.9) and total neurite length per neuron (61.3±3.3 μm vs 267.1±2.2 μm). All these parameters were brought down to control levels in the presence of Lorlatinib (1 μM) (neurites per neuron 8.7±1.9, number of branch points per neuron 3.6±0.5 and total neurite length per neuron 83±8.8 μm) (Control, n=19; ALKAL2, n=12 and ALKAL2+Lorlatinib, n=18 images from 3 independent experiments). (C) Resting membrane potential (RMP) of small DRG neurons in control (−56.06±1.03 mV), ALKAL2 (−55.15±2.21 mV) or ALKAL2+Lorlatinib (−54.5±1.53 mV) groups (n=19, 13 and 13, respectively). (D) Action potential (AP) threshold in control (−35.59±2.26 mV); ALKAL2 (−40.57±2.88 mV) and ALKAL2+Lorlatinib (−31.59±1.76 mV) groups (n=16, 11 and 9, respectively). (E) Representative AP discharge evoked by 100, 200, 300 and 400 pA current injections (1 s) in control, ALKAL2 (10 nM for 16 h.) and ALKAL2+Lorlatinib treated (10 nM and 1 μM for 16 hrs.) DRG neurons. (F) Measure of AP frequency evoked by current injection in the different groups represented in E (n=13, 11 and 9, respectively). Results indicate the mean±SEM. Statistical analysis was performed using one-way ANOVA (B) followed by Bonferroni post hoc test (**p<0.01) and paired (acute treatment) and unpaired t-test (C, D and F) (**p<0.01).

FIG. 5. ALKAL2 induces pALK in the dorsal horn of the spinal cord. (A) Intrathecal administration of ALKAL2 (1 μM) promotes thermal hyperalgesia, which is reversed by the administration of Lorlatinib (n=6 and 5, respectively). (B) Representative confocal image illustrating the pALK induction in the lamina I and II of the spinal dorsal horn following intrathecal infusion of ALKAL2. Activation of pALK is reversed by administration of Lorlatinib, prior to ALKAL2 administration. (C) Whisker plot of the pALK signal intensity represented in B) in the spinal dorsal horn (n=84, 56, 20 and 48 images from 4 mice for control; ALKAL2; ALKAL2+Lorlatinib 30 min and ALKAL2+Lorlatinib 1 h, respectively). Results indicate the mean±SEM. Statistical analysis was performed using one way ANOVA (C) followed by Bonferroni post hoc test (***p<0.001; ****p<0.0001).

FIG. 6. ALKAL2 gene deletion reverses inflammatory pain. (A) Schematic illustrating the experimental protocol of ALKAL2 oligodeoxynucleotide injection in the CFA pain model. (B) Western blot of ALKAL2 in lumbar DRG lysates at D9 following injection of ALKAL2 or scrambled ODN, compared to naïve control mice. (C) Whisker plot illustrating the reduction in ALKAL2 protein expression in the ODN treated animals (n=4-6 mice). (D) Representative confocal image of ALKAL2 immunostaining in DRG sections from control, ALKAL2, or scrambled ODN treated animals. (E) Measure of thermal withdrawal latency in contralateral and ipsilateral hind paws of CFA injected animals that received saline control, scrambled or ALKAL2 ODN (n=8, 7 and 8, respectively). Results indicate the mean±SEM. Statistical analysis was performed using one way ANOVA (C) followed by Bonferroni post hoc test (**p<0.01) and Two-way ANOVA (E) (**p<0.01; ***p<0,001).

FIG. 7. Pharmacological blockade of ALK receptor induces antinociception in acute and chronic preclinical pain models. (A) Shortening of the nociceptive behavior duration produced by intraplantar formalin (20 μl of 1.25% solution) after administration of increasing doses of Lorlatinib by gavage; vehicle was administered as control (n=6). (B) Paw withdrawal latency, in response to a thermal stimulus, in the CFA model, showed the anti-nociceptive effect of Lorlatinib (n=10 PBS; n=10 CFA+vehicle; n=12 CFA+Lorlatinib 0.3 mg/kg and n=10 CFA+Lorlatinib 1 mg/kg). (C) Paw withdrawal threshold (PWT) to mechanical stimuli was evaluated for 19 days after nerve injury. Lorlatinib inhibited PWT when compared to vehicle only (n=10). Results indicate mean±SEM. Statistical analysis was performed using one way ANOVA (A) followed by Bonferroni post hoc test (**p<0.01, ***p<0.001). and Two-way ANOVA (B and C) followed by Bonferroni post hoc test (###p<0.001 vs PBS i.pl or sham; *p<0.05, **p<0.01, ***p<0.001 vs CFA+Veh or PSNI+Veh).

FIG. 8. Characterization of the TRPV1-pHluorin channel. (A) I-V curves of capsaicin (100 nM)-evoked TRPV1 current in HEK cells transfected with either TRPV1wild type (circles) or TRPV1-pHluorin (squares) obtained from a ramp between −100 mV and +100 mV from a holding potential of 0 mV. At −100 mV the peak inward current was −10.2±1.63 pA/pF for WT vs −7.33±2.91 pA/pF for TRPV1-pHluorin. At +100 mV the peak outward current was 62.82±11.72 pA/pF and 61.36±11.97 pA/pF, respectively (n=8 and 9). (B) I-V curves of TRPV1 current recorded from HEK cells transfected with either TRPV1 WT (circles) or TRPV1-pHluorin (squares) and exposed to either 22° C. (control) or 50° C. At 50° C., the peak inward current (recorded at −100 mV) was −20.49±6.67 pA/pF for WT and −16.14±3.84 pA/pF for TRPV1-pHluorin channel (n=7). The peak outward current (recorded at +100 mV) was 192.84±25.37 pA/pF and 124.09±18.16 pA/pF, respectively. (C) I-V curves of TRPV1 current recorded from HEK cells transfected with either TRPV1 WT (circles) or TRPV1-pHluorin (squares) exposed to either pH 7.4 (control) or pH 6.5. At pH 6.5, the peak inward current (recorded at −100 mV) was −7.29±2.59 pA/pF for WT vs −7.73±1.93 pA/pF for TRPV1-pHluorin (n=7). The peak outward current (recorded at +100 mV) was 51.45±7.23 pA/pF and 45.92±7.06 pA/pF, respectively. (D) Whisker plot showing the resting membrane potential (RMP) in DRG neurons from WT and TRPV1-pHluorin mice at steady state and after exposure to capsaicin (100 nM). There was no significant shift with a mean value at rest of −57.51±1.08 mV and 56.51±0.74 mV (n=14), respectively, and after application of capsaicin −36.61±1.83 mV and −38.01±2.17 mV, for WT and pHluorin, respectively. (E) Whisker plot showing the action potential (AP) threshold in DRG neurons from WT and TRPV1-pHluorin mice. The values obtained were not different: −32.58±2.74 mV and −33.89±3.29 mV, respectively (n=12). (F) AP frequency evoked by 100, 200, 300 and 400 pA current injections (1 s) in DRG neurons from WT (circles) and TRPV1-pHluorin (squares) animals in the absence and presence of capsaicin (100 nM) (control conditions: 7.22±1.77 Hz in WT (n=9) vs. 2.44±6.67 Hz (n=11), respectively, and after capsaicin: 18.56±2.44 Hz in WT vs. 17.22±0.84 Hz evoked by the injection of 400 pA of current). Results indicate mean±SEM. Statistical analysis was performed using unpaired t-test and Two-way ANOVA.

FIG. 9. Characterization of the TRPV1-pHluorin expressing neurons. (A) Co-immunostaining of GFP (left) with markers of peptidergic (CGRP), non-peptidergic (IB4), C-LTMR (TH) neurons (middle) in DRG section. (B) Bar graph summarizing the results in (A) shows that most (˜60%) of the GFP+ neurons are peptidergic. (C) qRT-PCR analysis of selected genes (TRPV1, MOR, Tac1, TH, Lpar3, MrgprD) from GFP+ and GFP− neurons. Results indicate mean±SEM.

FIG. 10. ALKAL2 is not found in the human spinal cord. Representative RNAscope image showing the absence of ALKAL2 expression in human spinal cord.

FIG. 11. ALKAL2 is involved in acute pain. (A) Intrathecal ALKAL2 administration induces thermal hypersensitivity which is back to baseline 72 hrs after administration. The time-course of thermal hypersensitivity is dose-dependent (n=5 each group). (B) Intraplantar CFA enhanced pALK signal in the ipsilateral (CFA injected) spinal dorsal horn. On the right, whisker plot showing the fold change in pALK signal between ipsi and contralateral dorsal horn after intraplantar saline of CFA (n=22 and 19, respectively, from 3 animals each). Results indicate mean±SEM. Statistical analysis was performed using unpaired t-test (*p<0.05) and Two-way ANOVA followed by Bonferroni post hoc test (**p<0.01).

FIG. 12. Crizotinib reverses acute and chronic post-inflammatory hyperalgesia. (A) Administration of increasing doses of Crizotinib (i.t.) significantly shortened the nociceptive behavior duration after administration of formalin (20 μl of 1.25% solution intraplantar), when compared to vehicle (n=6). (B) The changes in paw withdrawal latency after thermal stimulus, at 3 days after administration of CFA, showed the anti-nociceptive effect of Crizotinib (n=7 PBS; n=7 CFA+vehicle; n=8 CFA+Crizotinib 30 ng and n=9 CFA+Crizotinib 100 ng). Results indicate mean±SEM. Statistical analysis was performed using one way ANOVA followed by Bonferroni post hoc test (A) (**p<0.01, ***p<0.001) and paired t-test for acute treatment (B); *p<0.05, **p<0.01, ***p<0.001). Two-way ANOVA (B) followed by Bonferoni post oc test (###p<0.01 vs PBS i.pl; *p<0.05, **p, <0.01 vs CFA+veh).

FIG. 13 Colitis was induced by adding 2.5% DSS in the drinking water (Water as control) of WT mice for 7 days. Body weight monitoring (A), colonic length (B) and macroscopic damage (C) of Water (n=4), DSS Veh (n=6) and DSS Lorlatinib (n=6) mice. Statistical analysis was performed using Two-way ANOVA followed by Tukey post hoc test (A) (**p<0.01, ***p<0.001, ****p<0.0001), One-Way ANOVA followed by Tukey post hoc test (B) (**p<0.01, ***p<0.001), or Kruskal-Wallis test followed by Dunn's post hoc test (*p<0.05) (C). (D) VMR to colorectal distension (CRD) in DSS colitis mice after treatment with Lorlatinib (1 mg/kg) or vehicle 1 h prior CRD (Water (n=4), DSS Veh (n=5) or DSS Lorlatinib (n=5)). Statistical analysis was performed using Two-way ANOVA followed by Tukey post hoc test (*p<0.05 vs DSS Lorlatinib).

FIG. 14 Colitis was induced by administration of 2.5% DSS (wt/vol) in drinking water for 5 days. On day 5, DSS was removed and replaced by water to allow mice to recover for 5 weeks. Body weight monitoring (A), colonic length (B) and macroscopic damage (C) of Water (n=6), post-DSS Veh (n=4-10) and post-DSS Lorlatinib (n=5-6) mice. Statistical analysis was performed using Two-way ANOVA followed by Tukey post hoc test (A) (*p<0.05, **p<0.01, ***p<0.001, ****p<0.0001) and Kruskal-Wallis test followed by Dunn's post hoc test (B-C). (D) VMR to colorectal distension (CRD) in post-DSS mice after treatment with Lorlatinib (1 mg/kg) or vehicle 1 h prior CRD (Water (n=6), post-DSS Veh (n=7) or post-DSS Lorlatinib (n=6)). Statistical analysis was performed using Two-way ANOVA followed by Tukey post hoc test (*p<0.05, ***p<0.001 vs post-DSS Lorlatinib). (E) ALKAL2 expression in lumbosacral DRG neurons of Water (n=6) and post-DSS (n=6). Statistical analysis was performed using t-test (*p<0.05).

### DETAILED DESCRIPTION

Generally, the present disclosure provides method of treating pain in a subject.

In one aspect, there is a provided a method of treating pain in a subject in need thereof comprising administering to the subject an anaplastic lymphoma kinase (ALK) inhibitor.

In one aspect, there is a provided a method of treating pain in a subject in need thereof comprising administering to the subject an ALKAL2 inhibitor.

The term “pain” as used herein refers to pain of any etiology, including acute and chronic pain, and any pain with an inflammatory component.

In some examples, pain includes, but is not limited to, inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer, including “break-through pain” and pain associated with terminal cancer, peripheral neuropathy and post-herpetic neuralgia.

The term “subject”, as used herein, refers to an animal, and can include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. In a specific example, the subject is a human.

The term “ALK’ refers to anaplastic lymphoma kinase.

In some examples, the ALK inhibitor is a small molecule, an antibody, a polynucleotide, or a pharmaceutical composition thereof.

In some examples, the ALKAL2 inhibitor is a small molecule, a pharmaceutical compositions, an antibody, or a polynucleotide.

The term “inhibit” or “inhibitor” as used herein, refers to any method or technique which inhibits protein synthesis, levels, activity, or function, as well as methods of inhibiting the induction or stimulation of synthesis, levels, activity, or function of the protein of interest, such as ALK and/or ALKAL2.

The term “small molecule” refers to organic compounds generally having a molecular weight less than about 1000, preferably less than about 500, which are prepared by synthetic organic techniques, such as by combinatorial chemistry techniques.

In a specific example, the ALK inhibitor small molecule is Lorlatinib or Crizotinib.

In a specific example, the ALKAL2 inhibitor is an antisense oligonucleotides (ODN) inhibiting the specific expression of ALKAL2.

In one example, the ALK inhibitor pharmaceutical composition comprises Lorlatinib or Crizotinib.

The term “polynucleotide” as used herein may be used interchangeably with “nucleic acid” and refers to polymers of nucleotides of any length, including ribonucleotides, deoxyribonucleotides, analogs thereof, or mixtures thereof. In some aspects, this term refers to the primary structure of the molecule. Thus, the term includes triple-, double and single-stranded deoxyribonucleic acid (“DNA), as well as triple-, double- and single-stranded ribonucleic acid (“RNA”). It also includes modified, for example by alkylation, and/or by capping, and unmodified forms of the polynucleotide.

The term “polypeptide”, which may be used interchangeable with “oligonucleotide”, refers to a polymer or oligomer of nucleotides, and may contain any combination of natural and non-natural nucleobases, sugars, and intenucleotidic linkages.

Polynucleotides may be single-stranded or double-stranded. A single-stranded oligonucleotide can have double-stranded regions (formed by two portions of the single-stranded oligonucleotide) and a double-stranded oligonucleotide, which comprises two oligonucleotide chains, can have single-stranded regions for example, at regions where the two oligonucleotide chains are not complementary to each other. Example oligonucleotides include, but are not limited to structural genes, genes including control and termination regions, self-replicating systems such as viral or plasmid DNA, single-stranded and double-stranded RNAi agents and other RNA interference reagents (RNAi agents or iRNA agents), shRNA, antisense oligonucleotides, ribozymes, microRNAs, microRNA mimics, supermirs, aptamers, antimirs, antagomirs, UI adaptors, triplex-forming oligonucleotides, G-quadruplex oligonucleotides, RNA activators, immuno-stimulatory oligonucleotides, and decoy oligonucleotides.

The term “antisense”, as used herein, refers to a characteristic of an oligonucleotide or other nucleic acid having a base sequence complementary or substantially complementary to a target nucleic acid to which it is capable of hybridizing. In some embodiments, a target nucleic acid is a target gene mRNA. In some embodiments, hybridization is required for or results in at one activity, e.g., an increase in the level of skipping of a deleterious exon in a target nucleic acid and/or an increase in production of a gene product produced from a target nucleic acid from which a deleterious exon has been skipped.

The term “antisense oligonucleotide” or “antisense polynucleotide”, as used herein, refers to an oligonucleotide complementary to a target nucleic acid. In some embodiments, an antisense oligonucleotide is capable of directing an increase in the level of skipping of a deleterious exon in a target nucleic acid and/or increase in production of a gene product produced from a target nucleic acid from which a deleterious exon has been skipped.

The term “identity” refers to the overall relatedness between polymeric molecules, e.g., between nucleic acid molecules (e.g., oligonucleotides, DNA, RNA, etc.) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes).

In one example, the ALK inhibitor antisense oligonucleotide is ODN1: 5′-GAAGCAGAGCGCACACAAAA (SEQ ID NO: 25); ODN2: 5′-TCCTCATCCATGGGCTCAGA SEQ ID NO: 26; ODN3: 5′-CGCTGAGGTTGAACTGGAGT SEQ ID NO: 27).

In one example, the ALKAL2 inhibitor antisense oligonucleotide is Design #1: ASO-1 DNA Sequence: 5′-AAGTGCTTGCTGCACTTCGG (SEQ ID NO: 1). Design #2: ASO-2 DNA Sequence: 5′-GATGGTGCAGTCTCTCGTGT (SEQ ID NO:2). Design #3: ASO-3 DNA Sequence: 5′-TGGTGTGTCGCTCCTTTGCA (SEQ ID NO:3).

In another example, the ALKAL2 inhibitor antisense oligonucleotide is Design #1: hASO-1 DNA Sequence: 5′-CAAGTACACTGATTTATCGA (SEQ ID NO:4). Design #2: hASO-2 DNA Sequence: 5′-GCACTACACGTCAAATGTGG (SEQ ID NO:5). Design #3: hASO-3 DNA Sequence: 5′-ACAATAGCTGGAATACTATT (SEQ ID NO: 6).

The term “polypeptide”, “peptide”, and “protein” may be used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term “antibody”, as used herein, encompasses an immunoglobulin whether natural or partly or wholly synthetically produced, and fragments thereof. The term also encompasses covers any polypeptide having a binding domain that is homologous to an immunoglobulin binding domain.

The term “antibody” further includes a polypeptide comprising a framework region from an immunoglobulin gene or fragments thereof that specifically binds and recognizes an antigen.

The term “antibody” may include whole antibodies, polyclonal, monoclonal and recombinant antibodies, fragments thereof, and further includes single-chain antibodies, humanized antibodies, murine antibodies, chimeric, mouse-human, mouse-primate, primate-human monoclonal antibodies, antiidiotype antibodies, antibody fragments, such as, e.g, scFv, (scFv)2, Fab, Fab′, and F(ab′)2, F(abl)2, Fv, dAb, and Fd fragments, diabodies, and antibody-related polypeptides. Antibody includes bispecific antibodies and multispecific antibodies so long as they exhibit the desired biological activity or function. In some aspects of the present disclosure, the biologically active molecule is an antibody or a molecule comprising an antigen binding fragment thereof.

In one example, the ALK inhibitor antibody is a monoclonal antibody, for example mAb30 or mAb49) (PMID: 15886198).

In one example, the inhibitor antibody is a monoclonal antibody, for example Mouse anti-Human ALK (Anaplastic Lymphoma Kinase)/CD246 Monoclonal Antibody.

A subject who is “susceptible to” or “at risk” of a disease, disorder, and/or condition, or symptoms or sequelae thereof, such as pain, has not been diagnosed with and/or does not exhibit symptoms of the disease, disorder, and/or condition but harbors a propensity to develop a disease or its symptoms.

The term “therapeutically effective amount” or “efficacious amount” refers to an amount of a compound, for example, that, when administered to a subject for treating pain, is sufficient to effect such treatment for the pain. The “therapeutically effective amount” will vary depending on the compound, the disease and its severity and the age, weight, etc., of the subject to be treated.

The term “pharmaceutically acceptable excipient”, “pharmaceutically acceptable diluent”, “pharmaceutically acceptable carrier”, and “pharmaceutically acceptable adjuvant” refers to an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use.

As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, a “pharmaceutical composition” refers to a composition suitable for administration to a subject, such as a mammal, especially a human. In general, a “pharmaceutical composition” is sterile, and generally free of contaminants that are capable of eliciting an undesirable response within the subject (e.g., the compound(s) in the pharmaceutical composition is pharmaceutical grade). are suitable for administration by a route other than transdermal administration.

As used herein, the term “pharmaceutically acceptable derivatives” of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and are either pharmaceutically active or are prodrugs.

A “pharmaceutically acceptable salt” of a compound, such as an ALK inhibitor or ALKAL2 inhibitor, means a salt that is pharmaceutically acceptable and that possesses the desired pharmacological activity of the parent compound. Such salts include: (1) acid addition salts, formed with inorganic acids such as hydrochloric acid,-hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

The term “treatment” or “treat” as used herein, refers to obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable.

The term treat “treating” and “treatment” may also mean prolonging survival as compared to expected survival if not receiving treatment.

The term “treating” and “treatment” as used herein also include prophylactic treatment.

It will be appreciated that in the treatment of pain, the effect may be prophylactic in terms of completely or partially preventing pain or a symptom thereof and/or can be therapeutic in terms of a partial or complete cure for pain and/or an adverse effect attributable to the disease.

The term “prevent” or “prevention” means no disorder or disease development if none had occurred, or no further disorder or disease development if there had already been development of the disorder or disease. Also considered is the ability of one to prevent some or all of the symptoms associated with the disorder or disease.

Methods of administration of the compounds and compositions of the present application include, but are not limited to, topical, intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal, vaginal and intestinal mucosa, etc.). Administration may be endoscopic or sublingual/buccal. Administration and may be administered together with other biologically active agents. Administration can be systemic or local.

In some examples, administration is topical administration. In another example, an inhibitor may be administered by topical administration. In another example, an inhibitor is formulated in a nanoparticle, and is for topical administration.

In some examples, topical administration of the nanoparticles or a composition comprising the nanoparticles may be to a surface of the subject such as the skin or mucous membranes such as the vagina, anus, throat, and ears. Passive topical administration refers to topical administration without the use of physical methods, such as mechanical and microporation methods, that disrupt the barrier properties of the stratum corneum and/or mucous membranes. The term active topical administration refers to administration of the nanoparticles or a composition comprising the nanoparticles with the use of physical methods, such as mechanical and/or microporation methods, that disrupt the barrier properties of the stratum corneum and/or mucous membranes.

The pharmaceutical compounds or compositions of the invention may be administered into the central nervous system by any suitable route.

The pharmaceutical compositions and formulations herein may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

In a specific example, treatment comprises administration of a therapeutically effective amount of a pharmaceutical composition to the central nervous system (CNS) of a subject.

Means of delivery to the CSF and brain include, but are not limited to intrathecal (IT), intracerebroventricular (ICV), and intraparenchymal administration. IT or ICV administration may be achieved through the use of surgically implanted pumps that infuse the therapeutic agent into the cerebrospinal fluid.

Intraparenchymal delivery may be achieved by the surgical placement of a catheter into the brain. As used herein, “delivery to the CSF” and “administration to the CSF” encompass the IT infusion or ICV infusion of GM1 through the use of an infusion pump. In some embodiments, IT infusion is a suitable means for delivery to the CSF

For intrathecal administration, the catheter is surgically intrathecally implanted.

As used herein, a “dosing regimen” or “therapeutic regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount.

Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Furthermore, “and/or” where used herein is to be taken as specific disclosure of each of the two specified features or components with or without the other. Thus, the term “and/or” as used in a phrase such as “A and/or B” herein is intended to include “A and B”, “A or B”, “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

### Abstract

The anaplastic lymphoma kinase (ALK) is a receptor tyrosine kinase known for its oncogenic potential and involved in the development of the peripheral and central nervous system. ALK receptor ligands, ALKAL1 and ALKAL2 were recently found to promote neuronal differentiation and survival. Here we show that inflammation or injury enhanced Alkal2 expression in a subset of TRPV1⁺sensory neurons. Notably, ALKAL2 was particularly enriched in both mice and human peptidergic nociceptors, yet weakly expressed in non peptidergic, large diameter myelinated neurons or in the brain. Using a co-culture expression system, we found that nociceptors exposed to ALKAL2 exhibit heightened excitability and neurite outgrowth. Intraplantar Complete Freund's adjuvant (CFA) or intrathecal infusion of recombinant ALKAL2 leads to ALK phosphorylation in the lumbar dorsal horn of the spinal cord. Finally, deletion of Alkal2 in dorsal root ganglia or blocking ALK with clinically available compounds Crizotinib or Lorlatinib, reversed thermal hyperalgesia and mechanical allodynia induced by inflammation or nerve injury, respectively. Overall, our work uncovers the ALKAL2-ALK signaling axis as a central regulator of nociceptor-induced sensitization. We propose that clinically approved ALK inhibitors used for Non-Small Cell Lung Cancer and neuroblastomas, could be repurposed to treat persistent pain conditions.

### Introduction

Sensitization of nociceptive neurons can lead to persistent pain in response to inflammation or injury. Identifying the mechanisms of peripheral sensitization has been key to defining the maladaptive long-lasting changes in nociceptive circuits which ultimately precipitate the transition to chronic pain (1-3). During inflammation, this sensitization process occurs particularly in thinly myelinated A6 and unmyelinated C-fibers that express the Transient Receptor Potential Vanilloid 1 (TRPV1) channel (2-5) and transduce inflammatory stimuli. Despite characterization of a large number of inflammatory mediators, their receptors and downstream signaling pathways, very few of these targets have led to efficacious treatments for pain relief (6, 7).

In the search of new therapeutics, targeting the Receptor Tyrosine Kinases (RTKs) family has shown promise for persistent pain conditions (6-11). Two members of the RTK family are the leukocyte tyrosine kinase (LTK) and the anaplastic lymphoma kinase (ALK) that belong to the insulin receptor family (12-15). Both members of this subfamily have been reported to promote cellular differentiation and growth, yet the physiological role of ALK has remained unclear. Initially described for its oncogenic properties (such as large-B cell lymphoma, non-small cell lung carcinoma (NSCLC) or neuroblastoma (16, 17), recent studies have highlighted ALK function in the developing nervous system (18-20).

As aberrant activation of ALK plays an important role in malignancy, research has focused on developing specific inhibitors for cancer therapy, and many of them are now used in clinical practice (21-23). Crizotinib (Xalkori, Pfizer) represents the first generation of FDA approved ALK inhibitor. However, Crizotinib had poor specificity, acting on ROS proto-oncogene 1-encoded kinase (ROS1), and was unable to penetrate the blood-brain barrier. More recently, the third-generation molecule Lorlatinib (PF-06463922) was introduced to overcome Crizotinib resistance. Lorlatinib has higher potency than previous generations of ALK inhibitors and shows good penetration of the blood-brain barrier (24).

As a classical RTK, ALK is composed of an extracellular domain, a single transmembrane domain, and a cytoplasmic domain that phosphorylates and activates multiple intracellular signaling pathways driving neuronal differentiation (25, 26). Recently, two novel proteins: family with sequence similarity 150A (FAM150A or ALKAL1) and family with sequence similarity 150B (FAM150B, ALKAL2), were identified as high affinity agonists of ALK/LTK receptors (26-28). Interestingly, ALKAL1 and ALKAL2 display homology only with one another and appear in several mammalian tissues including rat nerves where they induce the rapid formation of processes (14, 26, 27). Although both proteins physically interact with ALK, ALKAL1 shows greater affinity for LTK and ALKAL2 towards ALK (27, 28).

Here we identified ALKAL2, the ligand for the receptor tyrosine kinases ALK, as a biomarker of inflammation-induced nociceptor sensitization. We examined its pronociceptive properties and tested the analgesic efficacy of the clinically available ALK inhibitors Crizotinib and Lorlatinib in mouse models of inflammatory and neuropathic pain.

### Results

### ALKAL2 is Upregulated in TRPV1 Nociceptors During Inflammation

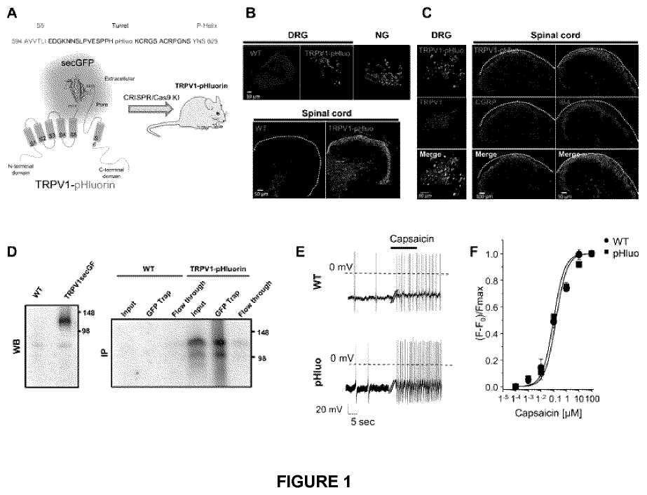

TRPV1 expression has been described in non-peptidergic and myelinated nociceptors earlier during development (29). Therefore, rather than using a genetic approach that labels neurons of the TRPV1 lineage, we opted for using a TRPV1-pHluorin knock-in mouse to identify the differentially expressed genes in TRPV1 nociceptive neurons. In this mouse, TRPV1 was conjugated to the super-ecliptic pHluorin inserted into the extracellular turret region of the channel (FIG. 1A) (30).

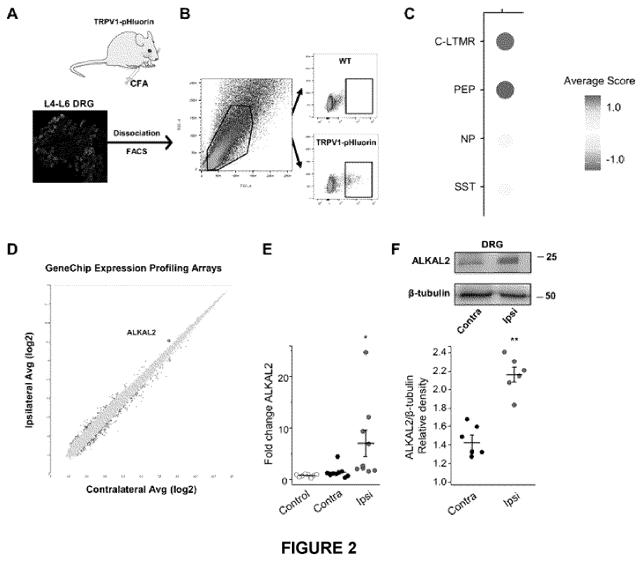

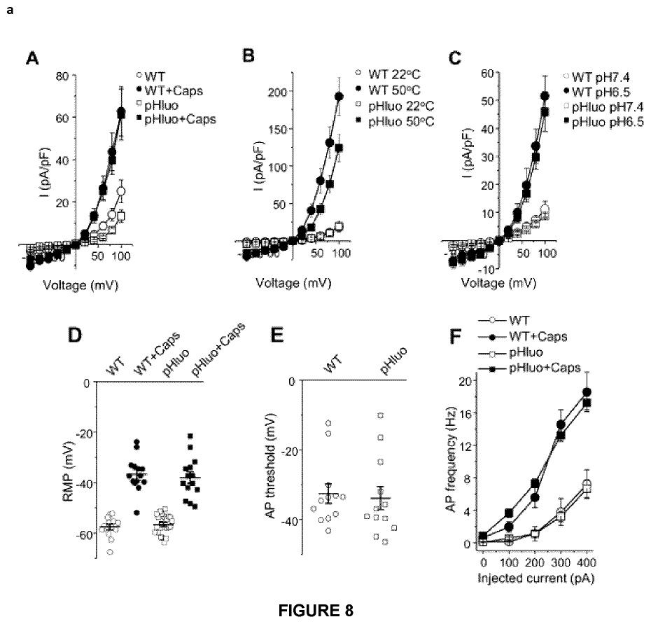

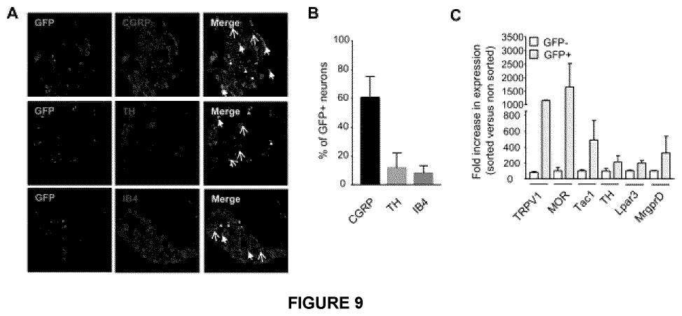

Insertion of pHluorin was previously found to not compromise the expression, trafficking and the sensitivity of the channel to capsaicin (31, 32). In addition, heat, and pH sensitivity of TRPV1-pHluorin DRG neurons were identical to WT channel (FIG. 8). We next examined the expression of TRPV1-pHluorin in the Dorsal Root and the Nodose/Jugular ganglia (FIG. 1B); and in central terminals of nociceptors located in the superficial dorsal horn of the spinal cord (FIGS. 1B and C). The identity of TRPV1+ neurons was verified by co-labeling analysis with calcitonin-gene related peptide (CGRP) (peptidergic nociceptors), IB4 (non-peptidergic), and TH (C-LTMR) (FIG. 9). The number of TRPV1′ neurons in DRGs and the innervation pattern in the spinal dorsal horn (FIGS. 1B and C) were not altered in the TRPV1-pHluorin mouse. Moreover, TRPV1-pHluorin protein could be immunoprecipitated from DRG tissue extracts, using GFP beads that recognize the pHluorin variant of GFP (FIG. 1D). We then validated the functional expression of TRPV1-pHluorin, previously reported in heterologous expression systems (33), in mouse DRGs. Capsaicin evoked action potential firing in pHluorin-labeled neurons (FIG. 1E), with a similar EC50 relative to WT channel (FIG. 1F). Next, we performed FACS purification of TRPV1-pHluorin neurons isolated from adult mice DRGs pooled from the lumbar region, using a gating of GFP^{high} cell population (FIGS. 2A and B). RNA was extracted from GFP(+) and GFP(−) neurons, PCR was run for specific markers of TRPV1 nociceptors (34), including TRPV1, MOR, or Tac1 that were found to be enriched in GFP(+) TRPV1 neurons, relative to GFP(−) neurons (FIG. 9C). These results indicate that the molecular signature and neuroanatomical organization of TRPV1 neurons is conserved in TRPV1-pHluorin knock-in mouse. As the DRG contains satellite cells, resident macrophages, and neurons, cell sorting of TRPV1+ neurons was verified by comparing the expression of the top 200 selected genes of the array to the single cell RNAseq data from Renthal et al (35). As shown in FIG. 2C, the score of expression assigned to each neuronal population indicates that these genes belong to the peptidergic nociceptor population, validating the isolation method illustrated in FIG. 2B.

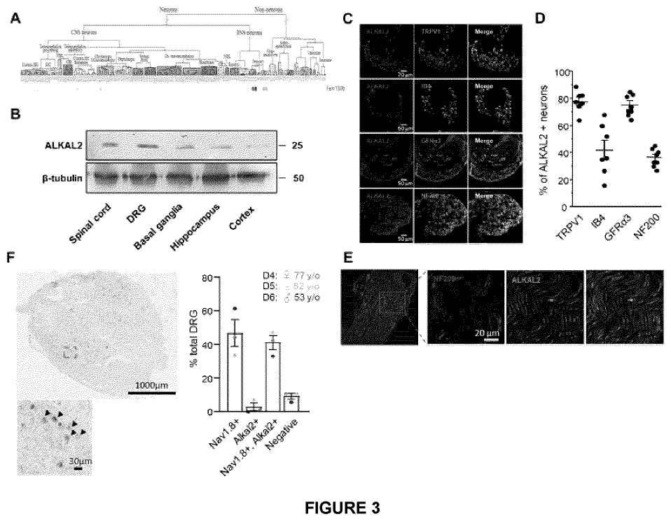

To identify putative genes that could contribute to the phenotypic plasticity of TRPV1 nociceptors in inflammation-induced sensitization, we used the Complete Freund's Adjuvant (CFA) model of chronic inflammatory pain. Three days post intraplantar injection of CFA, ipsilateral and contralateral lumbar (L4-L6) DRG neurons were separated, and TRPV1-pHluorin neurons were purified by FACS and analyzed through the GeneChip™ Mouse Gene 2.0 ST Array. The array analysis provided a complete expression profile of mRNA between contralateral and ipsilateral TRPV1 neurons following inflammatory insult (FIG. 2D). In inflammatory condition, we found >100 genes to be up or down regulated in TRPV1 neurons (Table 1), and among them, we noticed an increase in ALKAL2 (FAM150B) when compared to the contralateral side (FIG. 2D). Using qPCR, we measured a ˜7-fold increase in ALKAL2 mRNA from CFA-injected ipsilateral DRGs relative to contralateral or control DRGs from PBS injected mice (FIG. 2E). We next assessed ALKAL2 protein levels, using an epitope-directed antibody (New England Peptide/Vivitide). A band with a MW of ˜22 kDa (expected size ˜15 kDa) was detected by Western blot analysis from DRG lysates (FIG. 2F). Importantly, production of ALKAL2 was enhanced in the ipsilateral DRGs of CFA-injected mice (FIG. 2F). Evaluation of ALKAL2 expression in mouse single-cell RNA-sequencing data set from the Linnarsson's lab (http://mousebrain.org/genesearch.html) (36) identified ALKAL2 in the majority of peptidergic neurons: PEP1>PEP2>NP3>NP2>NP1>TRPM8^{high} (FIG. 3A). To confirm the transcriptomic data, we assessed and compared ALKAL2 protein level in the Cortex, Hippocampus, Basal ganglia, spinal cord and DRGs. Notably, the intensity of the signal was greater in the DRGs, compared to the CNS (FIG. 3B), confirming the high expression level of ALKAL2 in TRPV1-expressing sensory neurons.

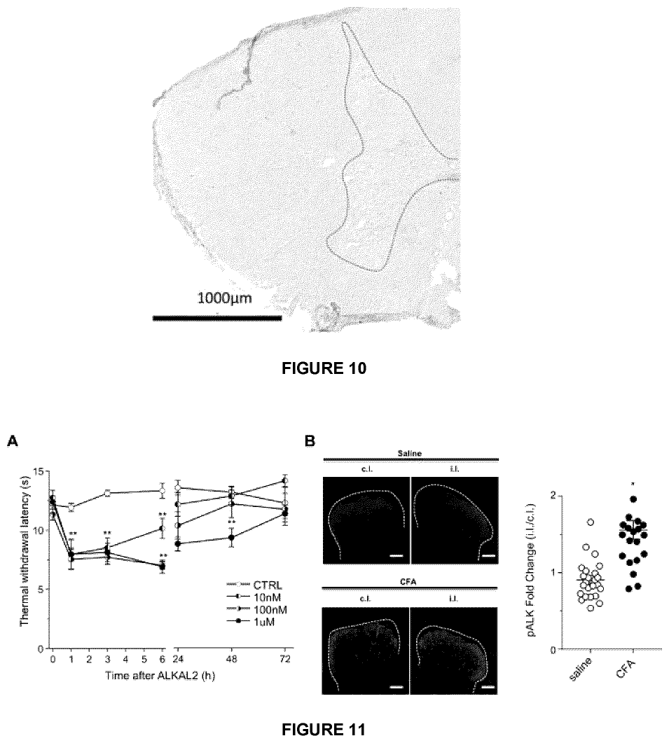

We next examined ALKAL2 protein in different subpopulations of DRG neurons: TRPV1, IB4 (non peptidergic), GFRα3 (peptidergic) and NF200 (large, myelinated neurons). As suggested by the transcriptomic and qPCR data, ALKAL2 was found in peptidergic TRPV1 neurons (˜80%), validating the microarray data of FIG. 2, and to a less extend in non peptidergic and NF200+ neurons (FIGS. 3C and D). Accordingly, in the sciatic nerve, ALKAL2+ fibers were clearly identified and did not overlap with myelinated NF200+ fibers, again confirming the expression of ALKAL2 in unmyelinated C fibers (FIG. 3E). Importantly, we also evaluated the expression of ALKAL2 in human DRG neurons and spinal cord by RNAscope. We used probes for mRNAs encoding Nav1.8 to identify nociceptors and found a high degree of colocalization between ALKAL2 and Na_{y}1.8+ nociceptors (FIG. 3F). In contrast, mRNA encoding ALKAL2 was not found in the human spinal cord (FIG. 10), indicative that it is selectively expressed and produced in afferent fibers.

### ALKAL2 Enhances Neurite Outgrowth and Excitability Via ALK

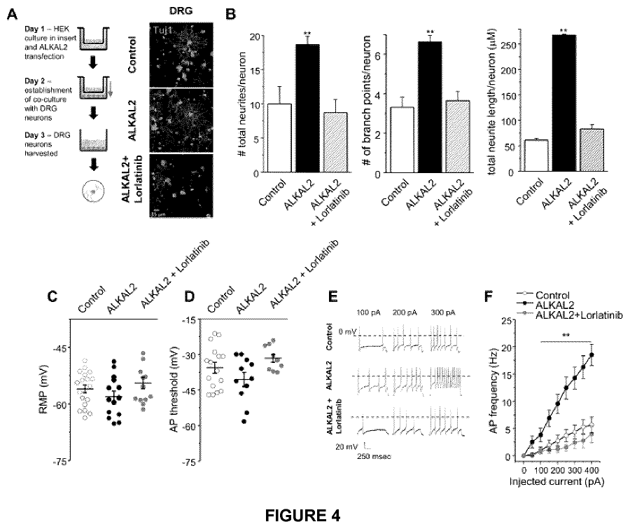

Previous work has shown that the ALK receptor plays a central role in the development of neural-crest derived cells and neurite outgrowth (18, 19, 37, 38). Using a co-culture system, we tested whether secreted ALKAL2 is able to activate ALK on DRG neurons, thus promoting neurite outgrowth through an autocrine/paracrine signaling process. In this assay, HEK cell monolayers seeded in the upper chamber of a transwell were transfected with ALKAL2, and then placed above acutely dissociated DRG neurons seeded into the lower chamber (FIG. 4A). DRG neurons were exposed to HEK-secreted ALKAL2 for a further 16 hours, after which, neurite growth was assessed on small and medium size neurons (FIG. 4A).

In the ALKAL2 transfected condition, we measured a two-fold increase in the total number of neurites (both primary and secondary branches) as well as branching points, and a five-fold increase in the total neurite length per neuron (FIG. 4B). Pharmacological inhibition of ALK with Lorlatinib was able to block ALKAL2-mediated neurite outgrowth, supporting a central role of the ALKAL2-ALK signaling in neuroplastic changes underlying hyperalgesia during inflammation.

To determine whether ALKAL2-mediated neurite growth was associated with neuronal hyperexcitability, we assessed the electrophysiological properties of TRPV1 neurons in response to ALKAL2. Neurons exposed to recombinant ALKAL2 for 16 h did not exhibit a change in their resting membrane potential or their action potential threshold (FIGS. 4 C and D). However, ALKAL2 induced a pronounced increase in the AP frequency evoked by ascending steps of injected current (FIGS. 4 E and F), and this effect was reversed by adding Lorlatinib into the DRG cell culture media. This finding suggests that ALKAL2 can promote neurite elongation along with hyperexcitability of TRPV1 neurons through an ALK receptor-mediated mechanism.

### Activation of ALK by ALKAL2 Induces Hyperalgesia

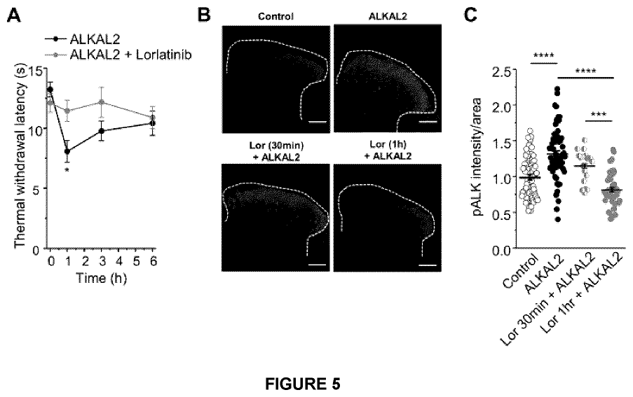

To investigate whether ALKAL2 could induce pain in vivo, we administered recombinant ALKAL2 by intrathecal injection, thereby targeting DRG and spinal cord neurons. In naïve mice, ALKAL2 induced thermal hyperalgesia lasting several hours in a dose-dependent manner (FIG. 11A). At a concentration of 1 μM ALKAL2, thermal hyperalgesia was blocked by a co-administration of Lorlatinib (FIG. 5A). Immunohistochemical analysis of the activated receptor in the spinal dorsal horn showed an increase in pALK, 30 min following ALKAL2 injection (FIGS. 5B and C). Notably, activation of ALK extends dorsally to the superficial lamina I that responds to noxious stimuli, and lamina II projection neurons that contribute to mechanical allodynia (39) (FIG. 5B). Importantly, oral administration of Lorlatinib, 30 min or 1 h, before ALKAL2 injection, reduced and completely blocked ALK receptor activation respectively (FIGS. 5B and C). Thus, ALKAL2 is able to induce ALK activation in the spinal cord, which leads to hyperalgesia.

Next, we assessed the activation of the ALK receptor in the inflammatory pain model. Intraplantar CFA enhanced pALK signal in the ipsilateral (CFA injected) spinal dorsal horn, whereas no changes were observed in saline injected mice (FIG. 11B). Importantly, the pattern of ALK activation in the superficial laminae I and II, was similar to the one obtained in response to ALKAL2 injection. Thus, ALKAL2-induced hyperalgesia is mediated by the activation of ALK in the spinal dorsal horn.

### ALKAL2 Deletion or Pharmacological Inhibition of ALK Prevents Persistent Inflammatory and Neuropathic Pain

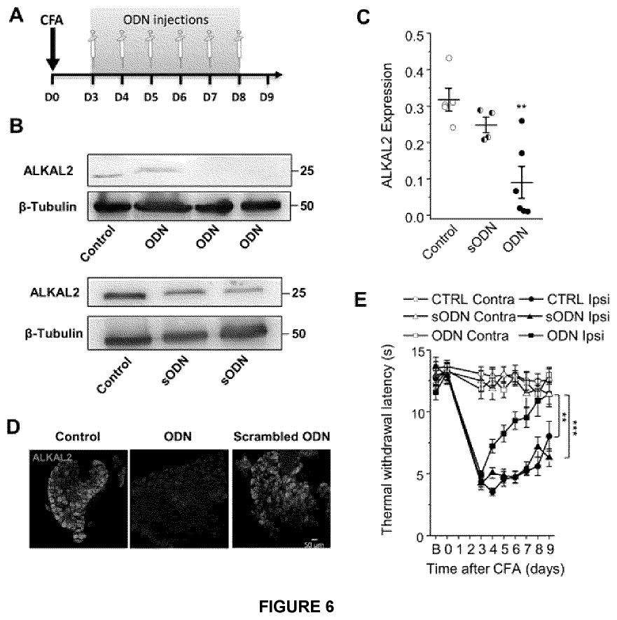

To test if the production of ALKAL2 by TRPV1+ primary afferents may lead to the activation of ALK, and subsequent inflammatory hyperalgesia, we first deplete ALKAL2 expression locally in the DRGs of CFA treated mice, using Alkal2 antisense oligonucleotides (ODN). Intrathecal injection of ALKAL2 ODNs between day 3 and 8 of CFA (FIG. 6A), significantly reduced ALKAL2 protein assessed at 8-day post injection, in lumbar DRG lysates or tissue sections (FIG. 6B-D). The long-lasting deletion of ALKAL2 induced antinociception, indicated by a faster recovery of thermal hyperalgesia in ALKAL2 ODN treated mice relative to scrambled controls (FIG. 6E). Together, these results demonstrate the pronociceptive action of ALKAL2 and hence suggest a central role of ALK in persistent pain.

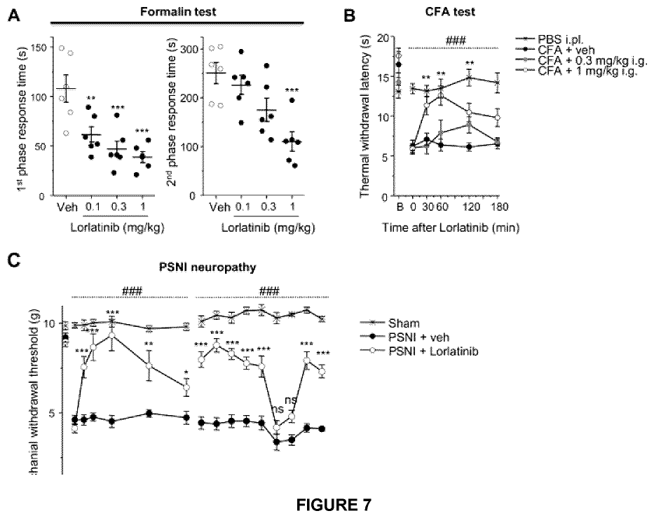

To test this, we investigated whether blocking ALK receptors in vivo is able to alleviate persistent pain induced by inflammation or nerve injury. A wide range of ALK inhibitors have proven to be safe in clinical trials and are now used in the treatment of malignancy (21-23). We started with the formalin test which captures mechanisms that are relevant to many clinical pain conditions, including the poorly localized, burning and throbbing pain sensation (33). The formalin test triggers two phases of nociceptive behaviors; the first is directly linked to the stimulation of the primary sensory neurons followed by a second phase associated with inflammation-induced sensitization. As shown in FIG. 7A, the orally available ALK inhibitor Lorlatinib, significantly inhibited nociceptive scores, in a dose-dependent manner, in both phases of the formalin test (78±3% in the first phase vs 56±4% in the second phase, n=6). Notably, the maximum dose used was only 1 mg/kg which is well below the therapeutic dose of 100 mg used for the treatment of malignancies (21).

Next, using the CFA-induced model of chronic inflammatory pain we assessed the antinociceptive effect of Lorlatinib. The highest dose of Lorlatinib 1 mg/kg, induced a transient but significant increase in paw withdrawal latency (60±5%, n=8) rapidly after administration (30 min) and lasting 2 hours (FIG. 7B), indicating an antinociceptive effect of Lorlatinib following CFA inflammation.

Finally, we used the partial sciatic nerve injury model, to test the ALK inhibitor on mechanical allodynia. Lorlatinib reduced the PWT within 30 minutes of daily administration, and the antinociceptive effects lasted throughout the entire testing period (19 days) (82±5% inhibition, n=10), demonstrating an absence of tolerance to Lorlatinib in neuropathic pain conditions (FIG. 7C).

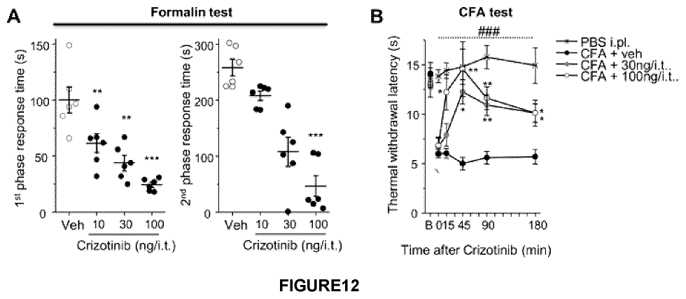

To confirm the effect of ALK inhibition in the spinal dorsal horn, we also employed Crizotinib via intrathecal delivery, in the Formalin test and the CFA pain model. Crizotinib showed a similar pattern of antinociceptive properties, blocking nocifensive behaviors evoked by formalin and thermal hyperalgesia after CFA. However, in the formalin test, Crizotinib potency was comparable for the early and later phase (76±2% in the first phase vs 81±7% in the second phase, n=8); (FIG. S5A). Paw withdrawal latency in the CFA model was also inhibited fast after Crizotinib administration with a robust antinociceptive effect at 30 and 100 ng, and a partial reversal of the drug effect at 3 hours post injection (FIG. 12B).

### Discussion

The lack of treatment options for chronic pain conditions calls for a fundamental reappraisal of the molecular mediators of nociception and sensitization. Recent work indicated the importance of Receptor Tyrosine Kinase, including TrkA and ErbB family (6, 7, 9) in persistent pain conditions. For instance, anti-NGF therapy has proven to be effective in reducing pain in osteoarthritis patients. Furthermore, the clinically approved EGFR inhibitors, Gefitinib and Lapatinib, were found to be analgesic in mouse models of inflammatory and neuropathic pain, whereas activation of the EGFR by its ligand epiregulin could enhance pain perception (9, 40). Here, we describe another neuronal RTK that is implicated in hyperalgesic priming. To faithfully capture the inflammation-mediated regulation of gene expression in TRPV1 primary afferents, we took advantage of a pHluorin-tagged TRPV1 knock-in mouse, rather than a TRPV1-Cre that labels a wider population of TRPV1-lineage neurons (41). We report that ALKAL2, the physiological ligand of ALK, is enriched in TRPV1 nociceptors. Chronic inflammatory pain enhances neuronal ALKAL2 expression and protein levels. Notably, ALKAL2 appears to be restricted to the peripheral nervous system as low level of proteins were found in different brain regions, including the cortex, hippocampus, basal ganglia. In cultured DRG neurons, we demonstrated that ALKAL2 reprogrammed TRPV1 nociceptors in an ALK dependent fashion. This transcriptional regulation leads to morphological and phenotypic changes highlighted by a slight shift in the threshold of spike initiation (not statistically different) and an increase in the action potential frequency. Importantly, Lorlatinib suppressed the hyperexcitability of ALKAL2-sensitized neurons, suggesting a beneficial effect of ALK tyrosine kinase inhibitors in hyperalgesic priming. Finally, the observed effects of ALKAL2 on cultured DRG neurons indicated that inflammation-induced production of ALK ligand may in turn engage ALK signaling via an autocrine way.

Although our work uncovered a role of ALKAL2 in neuroplasticity and sensitization of primary afferent neurons, our study does not address what mediators or receptor pathways drive ALKAL2 expression following CFA-induced inflammation. Several pattern recognition receptors transducing pathogen-associated molecular patterns (PAMPs) have been found in DRGs (42, 43). Among them, TLR4 activated by lipopolysaccharide (LPS), TRL3 and STING, receptors of bacterial and viral nucleic acids, or TLR5 that binds flagellin may directly or indirectly (via innate immune cells) drive ALKAL2 expression in cutaneous peptidergic nerves. Along these lines, recent studies reported an upregulation of ALKAL2 expression in methyl mercury-induced neurotoxicity (44), which might point to ALKAL2 as a key alarmin molecule produced by nociceptors to not only regulate nociception but also other physiological functions that could extend to cellular metabolism or stress response.

To test whether activation of ALK could contribute to pathological pain, we used Crizotinib and Lorlatinib in nociceptive and neuropathic pain models. We showed that intrathecal Crizotinib at low dose significantly suppressed nocifensive behaviors, in the formalin test, and thermal hyperalgesia following CFA paw injection. Using Lorlatinib IP, we found a transient but robust anti-nociceptive effect, even at 1 mg/kg, below the recommended dose for the treatment of ALK-positive metastasis. These findings identify the ALKAL2-ALK, as a central signaling hub through which inflammation or injury induces nociceptive sensitization.

Previous work identified two ALK ligands, ALKAL1 and ALKAL2, that bind to the extracellular domain of the ALK receptor, leading to activation of a downstream signaling in cell culture models (26). Interestingly, the two ligands display ˜47% similarity with one another but not with other mammalian proteins (20-22), making the inhibition of ALKAL2-ALK interaction a potential specific therapeutic approach that might have limited adverse side effects. ALKAL2, also named “augmentor-α” (AUG-α) or FAM150b, was first described in the adrenal gland (26) and the retina (45), where its signaling through ALK and LTK receptors has been implicated in autoimmunity, neurodevelopment and cancer. Previous studies also reported that ALKAL2 was able to activate ALK in vivo and co-expression of ALKAL2 with ALK was found to promote neurite outgrowth in neuroblastoma cell lines (36). Here we show that ALKAL2-ALK signaling induced neurite elongation and branching in DRG neurons, pointing towards receptor targeting as a way to prevent neuroplastic changes associated with peripheral and central sensitization. Functional examination of DRG neurons by electrophysiology reveal that ALKAL2-ALK signaling increases neuronal excitability, thus likely eliciting sensitization at synapses between TRPV1 C-fibers and dorsal horn neurons. Further work will define whether ALK alters neuronal excitability through transcriptional or post-translational regulation of voltage gated dependent channels, as previously described with IGF receptor modulation of T-type calcium channels (37).

Prior to the identification of ALKAL2, pleiotrophin and midkine have been proposed to act as ALK ligand (46, 47). Interestingly, these secreted growth factors induce neurite outgrowth and mitogenic activity in fibroblasts, epithelial, and endothelial cells, raising questions about the potential role of ALK signaling in tissue inflammation and repair. Accordingly, findings from Zeng et al. have highlighted ALK in regulating the inflammatory signaling pathway of sepsis (40). While future studies are warranted to determine whether neuronal ALKAL2 participate in the inflammatory response, it is plausible that, upon activation, peptidergic TRPV1 nerve endings release ALKAL2 at the periphery, thus contributing to skin and mucosal host responses to infection or injury. First described as an oncogene, several mutations of ALK have been linked to tumorigenesis, including neuroblastomas and non-small cell lung cancer (48), highlighting the importance of ALK in cancer biology. Notably, ALK is a member of the insulin receptor superfamily (14) that also promotes neuronal differentiation and maturation. It is expressed throughout the nervous system, particularly during embryogenesis, which supports a role of the receptor in neurodevelopment (13-15, 29). Surprisingly, ALK deficient mice do not present major behavioral deficits besides thinness due to decreased triglycerides levels and resistance to diet-induced obesity (49). Our present findings point to the importance of ALKAL2-ALK signaling in nociception and provide further knowledge on the biological role of ALK.

Given the relationship between the sympathetic and nociceptive system, it will be important to determine whether upregulation of ALKAL2 in the DRG neurons promote sympathetically maintained pain (50) in the context of arthritis or neuropathic pain. Although the ALKAL2 sequence predicts a secreted protein, the modality of action and secretion of this neuronal factor remain to be elucidated. While our data report an activation of ALK in the spinal dorsal horn, suggesting a release of ALKAL2 through axonal transport in the C fibers, one can speculate that ALKAL2 is secreted at the cell soma to act on neighboring neurons and satellite glial cells in the DRG, in an autocrine and paracrine fashion. Finally, ALKAL2 may be released in an activity dependent manner in the skin and mucosal tissues that receive C-fibers.

Together, our work identified ALKAL2 as a master regulator of injury-induced peripheral sensitization. Our findings are consistent with a model wherein inflammation promotes the expression and secretion of ALKAL2 from TRPV1 nociceptors. ALKAL2 activates ALK via autocrine/paracrine fashion to elicit neuronal hyperexcitability along with neurite outgrowth. This phenotypic change drives persistent pain associated with inflammation or nerve injury. Inhibition of the ALKAL2-ALK signaling prevents nociceptor hyperexcitability, thereby suppressing pALK in the spinal dorsal horn and alleviating thermal hyperalgesia and mechanical allodynia. Therefore, our work identified ALK as a future candidate receptor for pain management. As clinical studies have indicated that Crizotinib or Ceritinib therapy could reduce chest, arm and shoulder pain in NSCL cancer patients (51-53), it will be important to confirm that ALK inhibition is analgesic in human patients.

TABLE 1
Differentially expressed genes. Upregulated and downregulated genes
with statistical significance and fold change between ipsilateral
(CFA) and contralateral groups. Significant genes were selected by
fold change (>2.0- or <−2.0-fold) and adjusted p-value (<0.05).

| | |
|---|---|
| Upregulated | Downregulated |

| | Fold | | Fold |
|---|---|---|---|
| Symbol | change | Symbol | change |
| ALKAL2 | 2.00 | n-R5s71 | −2.06 |
| Wfdc6a | 2.06 | n-R5s152 | −2.20 |
| Vmn1r20 | 2.04 | mt-Ti | −2.18 |
| Trbv13-1 | 2.02 | mt-Ta | −2.13 |
| Trav9n-4 | 2.13 | Vmn1r18 | −2.02 |
| Traj53 | 2.13 | Uts2b | −2.07 |
| Tmem173 | 2.22 | Tnnt2 | −2.50 |
| Tdpoz2 | 2.26 | Spata31d1b | −2.38 |
| Ssxb9 | 2.53 | Snord82 | −2.18 |
| Psg20 | 2.41 | Rps15 | −2.39 |
| Olfr784 | 2.07 | Psg22 | −2.19 |
| Nlrp5 | 2.06 | Prg4 | −2.19 |
| Mptx2 | 2.05 | Pln | −2.18 |
| Mir693 | 2.23 | Orm2 | −2.00 |
| Mir296 | 2.54 | Olfr988 | −2.54 |
| Mir1900 | 2.50 | Olfr610 | −2.54 |
| Mir1224 | 2.21 | Olfr591 | −2.08 |
| LOC10105595 | 2.01 | Olfr59 | −2.65 |
| 3 | 2.08 | Olfr494 | −2.15 |
| Itk | 2.42 | Olfr467 | −2.07 |
| Igkv4-80 | 4.20 | Olfr299 | −2.72 |
| Ighv1-80 | 2.02 | Olfr1356 | −2.00 |
| Ifnl3 | 2.42 | Nppa | −2.52 |
| Gpx2-ps1 | 2.13 | Naip6 | −2.61 |
| Gm9257 | 2.44 | Myoz2 | −2.05 |
| Gm8212 | 2.12 | Mir501 | −2.13 |
| Gm5862 | 2.14 | LOC1010562 | −2.11 |
| Gm5071 | 2.02 | 78 | −2.17 |
| Ifnl3 | 2.42 | I11r2 | −2.48 |
| Gpx2-ps1 | 2.13 | Igkv14-126 | −2.43 |
| Gm9527 | 2.44 | Ighv1-20 | −2.86 |
| Gm8212 | 2.12 | Igh-VJ558 | −2.29 |
| Gm5862 | 2.14 | Gm8247 | −2.45 |
| Gm5071 | 2.26 | Gm5565 | −2.91 |
| Gm25344 | 2.17 | Gm4963 | −2.35 |
| Gm24984 | 2.08 | Gm4825 | −2.21 |
| Gm24938 | 2.08 | Gm26251 | −2.02 |
| Gm24938 | 2.08 | Gm26036 | −2.05 |
| Gm24938 | 2.21 | Gm26016 | −2.80 |
| Gm24885 | 2.81 | Gm25408 | −2.04 |
| Gm24402 | 2.21 | Gm25102 | −3.89 |
| Gm24203 | 2.90 | Gm25008 | −2.34 |
| Gm23244 | 2.29 | Gm24282 | −2.30 |
| Gm22927 | 2.47 | Gm24149 | −2.29 |
| Gm22876 | 2.13 | Gm24117 | −2.48 |
| Gm22535 | 2.51 | Gm24009 | −2.00 |
| Gm22509 | 2.27 | Gm23741 | −2.62 |
| Gm20736 | 2.27 | Gm23201 | −2.10 |
| Gm20736 | 2.27 | Gm23102 | −2.17 |
| Gm20736 | 2.27 | Gm22883 | −2.17 |
| Gm20736 | 2.02 | Gm22831 | −2.17 |
| Gm20736 | 2.27 | Gm22749 | −2.02 |
| Gm20736 | 2.27 | Gm22740 | −3.07 |
| Gm20736 | 2.27 | Gm22739 | −2.35 |
| Gm20736 | 2.27 | Gm22602 | −2.21 |
| Gm20736 | 2.27 | Gm22558 | −2.10 |
| Gm20736 | 2.02 | Gm22341 | −2.02 |
| Gm17268 | 2.18 | Gm22301 | −2.18 |
| Gm16391 | −2.19 | Gm22289 | −2.40 |
| Gm16248 | 2.07 | Gm22007 | −2.02 |
| Gm15182 | 2.70 | Fabp4 | −2.03 |
| Gm15127 | 2.59 | Dsg1a | −2.05 |
| Gm11677 | 2.61 | Ctsj | −2.02 |
| Gm11084 | 2.20 | Cma2 | −2.04 |
| Gm10923 | 2.52 | C1ql1 | −2.25 |
| Gm10700 | 2.15 | Aspn | −3.05 |
| Fbxw21 | 2.26 | Apol11b | −2.10 |
| Bdnf | 2.00 | Adam34 | −2.52 |
| 1700093J21Rik | | Actc1 | −2.09 |
| | | 4930526F13Rik | |

TABLE 2
Primer sequences

| | | SEQ ID | | SEQ ID |
|---|---|---|---|---|
| Gene | Primer forward | NO | Primer reverse | NO: |
| ALKAL2 | TGAGGTTGCTAGTT | 7 | AGGAACAATCTCCA | 8 |
| | GAGCTGG | | CTCTCTGC | |
| GAPDH | CCATGGAGAAGGCT | 9 | CAAAGTTGTCATGG | 10 |
| | GGGG | | ATGACC | |
| TRPV1 | CAACAAGAAGGGGC | 11 | TCTGGAGAATGTA | 12 |
| | TTACACC | | GGCCAAGAC | |
| TH | CCCAAGGGTTCAGA | 13 | GGGCATCCTCGAT | 14 |
| | AGAG | | GAGACT | |
| MOR | GAGCCACAGCCTGT | 15 | CGTGCTAGTGGCT | 16 |
| | GCCCT | | AAGGCATC | |
| TAC1 | AAAGCAGGAGCTGG | 17 | TGTTCGATTTTGCG | 18 |
| | GGACTA | | GTCAGA | |
| LPAR3 | GTCTTAGGCGCCTT | 19 | TTGCACGTTACACT | 20 |
| | CGTGG | | GCTTGC | |
| MRGPRD | GGTGCTGCTGGAAA | 21 | TGGAGTTCATTCCT | 22 |
| | CACTTC | | GCTCCTG | |

### Materials and Methods

### Animals

We used adult male C57BL/6 J mice purchased from the Jackson Laboratory. All experiments were conducted on male age-matched animals, under protocols approved by the University of Calgary Animal Care Committee and in accordance with the international guidelines for the ethical use of animals in research and guidelines of the Canadian Council on Animal Care. Animals were housed at a maximum of three per cage (30×20×15 cm), with water and food ad libitum. They were kept in controlled temperature of 23±1° C. on a 12 h light/dark cycles (lights on at 7:00 a.m.) and all experiments were performed between 10 am and 3 μm. Different cohorts of mice were used for each test.

Ecliptic GFP (superecliptic pHluorin, or EcGFP) was inserted into the trpv1 gene at position 1842, between 615th and 616th residues (histidine and lysine, respectively)³⁷, using CRISPR/Cas9-mediated homology directed repair. Cas9 nuclease was guided to the sequence, GCAGATCCCCGACACTTGTG (SEQ ID NO: 23), which was cloned in pX330 plasmid. The hCas9 sequence from pX330 was cloned in RClscript-Goldy TALEN plasmid, replacing the sequence between Xenopus globin 5′- and 3′UTR. Both sgRNA and Cas9 mRNA were prepared by in vitro transcription, using MEGAshortscript Kit and mMESSAGE mMACHINE T3 Transcription Kit (Life Technologies), respectively. Single-stranded donor DNA, with 725 nt upstream and 850 nt downstream homology arm, was prepared using nicking endonucleases. A mixture of sgRNA, Cas9 mRNA, and single-stranded donor was injected into pronuclei of fertilized mouse eggs. PCR and sequence analysis of resulting mice identified a single mouse as carrier of correctly inserted EcGFP.

### Human Dorsal Root Ganglia Samples

Human DRG were obtained from three brain-dead organ-donor patients (61-76-year-old) under the approval of the French institution for organ transplantation (Agence de la Biomédecine, DC-2014-2420). The three patients died from stroke. Body temperature was lowered with ice, and blood circulation was maintained for 3 hours before vertebral bloc removal. After organ removal for transplantation purpose, a spinal segment from thoracic level (T9) to the caudal end was removed in one piece and spinal cord and DRGs were immediately dissected in ice cold oxygenated HBSS solution. Tissues were subsequently flash frozen in liquid nitrogen. This short time interval allowed good preservation of the tissue, as indicated by the near absence of morphologically altered cells as previously reported (54). For subsequent in situ hybridization experiments, frozen ganglia 12 μm sections were prepared with a cryostat and mounted on SuperFrost Plus (ThermoFisher).

### Intrathecal Drug Treatment

Intrathecal injections were performed in conscious mice. Briefly, mice were manually restrained, the dorsal fur of each mouse was shaved, the spinal column was arched, and a 30-gauge needle attached in a PE20 Polyethylene tube to a 25 μl Hamilton micro syringe (Hamilton, Birmingham, UK) was inserted into the subarachnoid space between the L4 and L5 vertebrae. Accurate positioning of the needle tip was confirmed by a characteristic tail-flick response of animal when the needle if correctly positioned. Intrathecal injections of 10 μl were delivered over a period of 5 seconds.

### Formalin Test

Formalin test was performed as originally described (33) and as routinely performed in our lab (55). Briefly, mice were acclimatized in the laboratory for at least 60 min. before experiments. Animals received 20 μl of formalin solution (1.25%) prepared in phosphate buffered saline (PBS) and injected in the plantar surface of the right hind paw (Intraplantar: i.pl). Following i.pl. injections of formalin, mice were immediately placed individually into observation chambers and the time spent licking or biting the injected paw was recorded and considered as nocifensive responses. We observed animals individually and measured nocifensive responses from 0 to 5 min (acute nociceptive phase) and 15 to 30 min (inflammatory phase). Crizotinib was delivered by intrathecal injection (i.t.) 20 min. prior testing. Lorlatinib was delivered either spinally (i.t.) or systemically (i.g.) 30 min prior testing.

### Persistent Inflammatory Pain Induced by CFA

To induce thermal hyperalgesia produced by peripheral inflammation, 20 μl of Complete Freund's Adjuvant (CFA) was injected subcutaneously in the plantar surface of the right hind paw (i.pl.) (56). Sham groups received 20 μl of PBS in the ipsilateral paw. Animals were treated with either Crizotinib (Millipore Sigma) delivered spinally (i.t.) or Lorlatinib (Millipore Sigma) systemically (i.g.) or vehicle (10 ml/kg) 3 days following CFA injection and their thermal withdrawal threshold were subsequently tested.

For the ALKAL2 deletion mice were treated with antisense oligonucleotides ODN, Alkal2 or scrambled (5 ug, i.t.; Integrated DNA Technologies), or vehicle control for 5 consecutive days at D3 post CFA injection. Thermal withdrawal threshold was assessed at 3, 4, 5, 6, 7, 8 and 9 days of CFA treatment. Design #1: ASO-1 DNA Sequence: 5′-AAGTGCTTGCTGCACTTCGG (SEQ ID NO: 1). Design #2: ASO-2 DNA Sequence: 5′-GATGGTGCAGTCTCTCGTGT (SEQ ID NO: 2). Design #3: ASO-3 DNA Sequence: 5′-TGGTGTGTCGCTCCTTTGCA (SEQ ID NO: 3). scrambled ODN: 5′-TGTGCTGCTTGTACTGGCCT (SEQ ID NO: 24).

### Thermal Hyperalgesia

Thermal hyperalgesia was examined by measuring the latency to withdrawal of the right hind paws on a focused beam of radiant heat (IR=30) of a Plantar Test apparatus (UgoBasile, Varese, Italy). Animals were placed individually in a small, enclosed testing arena (20 cm×18.5 cm×13 cm, length×width×height) on top of a wire mesh floor. Mice were allowed to acclimate for a period of at least 90 minutes. The device was positioned beneath the animal, so that the radiant heat was directly under the plantar surface of the ipsilateral hind paw. Three trials for each mouse were performed. The apparatus was set at a cut-off time of 30 s to avoid tissue damage. Thermal hyperalgesia was evaluated immediately prior to the treatments (Time 0) and 15, 45, 90 and 180 minutes after intrathecal treatment with Crizotinib or after 30, 60, 120 and 180 minutes of Lorlatinib administration. For ALKAL-2 induced hyperalgesia, mice were treated with ALKAL2 (0.01; 0.1; 1 uM, i.t.) or vehicle, after Lorlatinib administration (1 mg/kg, i.g.), and their thermal hyperalgesia was evaluated 1, 3, 6, 24, 48 and 72 hours post ALAKAL2 treatment.

### Partial Sciatic Nerve Injury-Induced Neuropathic Pain

Mice were anesthetized with isoflurane (5% induction, 2.5% maintenance). A partial ligation of the sciatic nerve was performed by tying the distal one third to one half of the dorsal portion of the sciatic nerve, according to the procedure described by (57). In sham-operated mice, the sciatic nerve was exposed without ligation. The wound was closed and covered with iodine solution. Fourteen days after surgery, mice were treated with Lorlatinib (1 mg/kg, i.p.) or vehicle (10 mg/kg, i.p.), while sham-operated animals received only vehicle (10 ml/kg, i.p.). Mechanical withdrawal thresholds were evaluated immediately before the surgeries (baselines), then 14 days after the surgeries (day 0) and at various time points (0.5, 1, 2, 3, 4, 6 h) after treatment and every 2 days afterwards.

### Evaluation of Mechanical Hyperalgesia

Mechanical hyperalgesia was measured using a dynamic plantar aesthesiometer (DPA; Ugo Basile, Varese, Italy) as routinely performed in our laboratory (55, 58). Animals were placed individually in small, enclosed testing arenas (20 cm×8.5 cm×13 cm, length×width×height) on top of a wire grid platform. Mice were allowed to acclimate for a period of at least 90 min. The DPA device was positioned beneath the animal so that the filament was directly under the plantar surface of the ipsilateral hind paw. Each paw was tested three times per session.

### Isolation of DRG Neurons

DRG neurons were harvested from adult mice and enzymatically dissociated in Hank's balanced salt solution (HBSS) containing 2 mg/ml collagenase type I and 4 mg/ml dispase (Invitrogen) for 45 min at 37° C. DRGs were rinsed twice in HBSS and once in Neurobasal A culture medium (Thermo Fisher Scientific) supplemented with 2% B-27, 10% heat-inactivated fetal bovine serum (HI-FBS), 100 μg/ml streptomycin, 100 U/ml penicillin, 100 ng/ml of Nerve Growth Factor (NGF) and 100 ng/ml glial cell-derived neurotrophic factor (GDNF) (all from Invitrogen). Individual neurons were dispersed by trituration through a fire-polished glass Pasteur pipette in 4 ml media and cultured overnight at 37° C. with 5% CO2 in 96% humidity on glass coverslips previously treated with 25% Poly-Ornithine and Laminin (both from Sigma).

For co-culture experiments, human embryonic kidney (HEK) 293 tsA-201 cells were grown to 80% confluence at 37° C. (5% CO₂) in Dulbecco's modified Eagle's medium (+10% fetal bovine serum, 200 units/ml penicillin and 0.2 mg/ml streptomycin (Invitrogen, Carlsbad, CA, USA)) in the transwell of a 12-well cell culture plate (Gibco). Cells were transfected with 0.5 or 1 μg of ALKAL2 plasmid (Genomics, ABIN3292379) using the calcium phosphate method and washed 12 hours after transfection. After another 8 hours, isolated DRG neurons plated on glass coverslips were placed in the bottom chamber of the 12-well plate and the media was changed to Neurobasal A culture medium (see above). Electrophysiological recordings or immunohistochemistry were conducted 24 hours later.

### FACS Analysis

At day 3 of CFA, ipsilateral and contralateral DRG tissue (L4-L6) from TRPV1-ecGFP mice were collected and digested separately. After digestion, cells were filtered through a 90 mm mesh (Sarstedt) and washed in PBS 1% FBS. Cells were stained for 20 min with anti-GFP antibody (Sigma) followed by anti-goat Alexa Fluor 488 antibody (Invitrogen) for another 20 min and analyzed on a FACS Aria II (BD Bioscience).

### Gene Expression Analysis

After FACS sorting, RNA was extracted separately from GFP positive and GFP negative cells using a RNeasy Mini kit (Qiagen), eluted in 20 ul of water with the eluate passed twice through the column to increase yield. The quantity of RNA was determined using a Nanodrop 2000c spectrophotometer (Thermo-Fisher Scientific). Three biological replicates for the GFP positive and GFP negative samples (at ˜50 ng/sample) were submitted to the Centre for Applied Genomics (Toronto). Here the quality of the samples was assessed using the Agilent Bioanalyzer 2100 with the RNA Pico chip kit (Agilent Technologies). RNA integrity number values between 6.5 and 7 were achieved. The expression profiling was performed according to the manufacturer's instructions with Affymetrix GeneChip Mouse Gene 2.0 ST Array (Affymetrix, Santa Clara, CA, USA). Primary data analysis was carried out with the Affymetrix Expression Console 1.4.1.46 software including the Robust Multiarray Average module for normalization. Gene expression data were log-transformed. A change was considered significant when the FDR-corrected p-value/q-value thresholds met the criterion q<0.01 at fold changes >2 (expression increments or declines larger than two).

Extraction and Analysis of Existing scRNAseq Dataset:

Single cell RNA sequencing data of mouse DRG from Renthal et al., (35) were downloaded from the GEO portal and processed with the Seurat R package (v 4.0.2). Single cell data were normalized with SCTransform function, then data were scaled on all features. Only cells identified as SST, NP, PEP or c-LTMRs in Renthal et al were used for analysis.

Comparison of Gene Expression with scRNAseq Dataset

After normalization of Affymetrix FPKM, only 19699 genes conserved between the two data sets were used for analysis. A list of the 200 most expressed genes (with the highest mean of FPKM) in the contralateral condition of the Affymetrix dataset from cell-sorted TRPV1-pHluorin samples was built. This list was then used to compute a score on Renthal scRNAseq dataset using the AddModuleScore function from Seurat R packages. Briefly, from the input list, Seurat calculates the average expression levels of each cluster (SST, NP, PEP or c-LTMRs) on a single cell level, subtracted by the aggregated expression of module features from a set of “matched” controls. All analyzed features (from the input list) are binned based on averaged expression, and the control features are randomly selected from each bin. A score is then assigned to each cell of the cluster based on the comparison between the average gene expression of the cluster computed as described above and the average gene expression within the cell.

### RNAscope In Situ Hybridization

RNAscope in situ hybridization multiplex assay was performed as instructed by Advanced Cell Diagnostics (ACD). PFA-fixed tissue was used, and slides removed from the −80° C. freezer were immediately washed with PBS (pH 7.4; 5 min, twice) and then dehydrated in 50% ethanol (5 min), 70% ethanol (5 min) and 100% ethanol (10 min) at room temperature. The slides were air dried briefly and then boundaries were drawn around each section using a hydrophobic pen (ImmEdge PAP pen; Vector Labs). When hydrophobic boundaries had dried, protease IV reagent was added to each section (30 min) until fully covered. Slides were washed briefly in PBS at room temperature and then placed in a prewarmed humidity control tray (ACD) containing dampened filter paper. The RNAscope assay was performed according to the manufacturer's instructions using a HybEZ oven (ACD). The probe used was Mm-Alkal2 (ACD, #531801). The slides were washed, and cover slipped with Prolong Gold Antifade mounting medium with DAPI (Invitrogen, Thermo Fisher).

### Biochemistry

Collected organs were homogenized using a bullet blender (Next Advance) with SSB02 beads (Next Advance) and lysed in RIPA buffer (0.1% SDS, 1% Triton X-100 and 0.5% Na deoxycholate in PBS (all from Sigma-Aldrich)) with Halt protease and phosphatase inhibitors (Thermo Scientific) for 45 minutes. Lysates were centrifuged at 10,000 g for 10 min at 4° C., supernatants were collected, and protein concentration was quantified and normalized using a Bradford assay (Bio-Rad Laboratories). Total lysates were separated by SDS-PAGE (7-10%) and transferred onto nitrocellulose membranes (Sigma-Aldrich). Membranes were blocked in 5% nonfat dry milk for 1 h at room temperature, and then probed with anti-ALKAL2 antibody (1/100 dilution in 5% milk: New England Peptides) at 4° C. overnight. Membranes were then washed three times with TBS-T and incubated with horseradish peroxidase (HRP)-conjugated anti-rabbit antibodies (1:1000; GE Healthcare) for 1 h at room temperature. Bands were visualized using the Immobilon Western chemiluminescent HRP Substrate (Bio-Rad), and band density was calculated using Image J. Intensity of Rabbit anti-Beta-tubulin III antibody (1/1000 dilution in 5% milk; Sigma-Aldrich) band was used for normalization among samples.

### RNA Extraction and RT-qPCR

DRGs were harvested at 72 h of intraplantar CFA injection, dissociated using a bullet blender (Next Advance) with SSB02 beads (Next Advance) in RLT buffer (Qiagen). Total RNA was extracted using a RNeasy Mini kit (Qiagen), according to the manufacturer's instructions. The quality and quantity of RNA were determined using a Nanodrop 2000c spectrophotometer (Thermo-Fisher Scientific). Relative Alkal2 gene expression (normalized to GAPDH) was determined by qPCR using BrightGreen PCR Master Mix (ABMgood) and a StepOnePlus real-time PCR detection system (Applied Biosystems). The designed primers for DNA amplification are listed in Table 2.

### Immunostaining and Confocal Microscopy.

Spinal cords and DRG (L4-L6) were collected from CFA-injected mice and were fixed for either 3 h (DRG) or 24 h (spinal cords) in 4% paraformaldehyde (PFA) (Sigma) followed by 24 hours treatment with 30% sucrose. Tissues were then embedded in either 3% agarose or OCT and cut at 9 μm section with a vibratome (Leica) for agarose or 10 μm section with a cryostat for OCT (Thermo-Fisher Scientific) embedded tissue, respectively, onto Superfrost slides (VWR International). Tissues were washed two times in phosphate-buffered saline (PBS) and then blocked for 60 min at room temperature with a PBS solution containing 3% Fetal Bovine Serum (FBS) and 0.3% Triton-X incubated overnight in PBS 3% BSA, 0.01% T-X100 at 4° C. with either polyclonal chicken-anti GFP (1/500, Invitrogen A10262), polyclonal rabbit-anti GFP (1/500, Chromotek, PABG1), or polyclonal rabbit-anti TRPV1 (1/500, Alomone, ACC-030). For pALK immunostaining, we used a polyclonal rabbit-anti pALK (1/100 Cell signaling #3341) in TBS solution containing 0.2% Triton-X100, 0.05% Tween 20 (TBS-T), and 5% Goat Serum+5% Donkey Serum (Sigma). After extensive wash in the blocking buffer, tissues were incubated for 1 h at RT with secondary antibodies (anti-chicken IgG conjugated to Alexa Fluor 488, anti-rabbit IgG conjugated to Alexa Fluor 488, anti-rabbit IgG conjugated to Alexa Fluor 555, all from Invitrogen) for 1 hour at room temperature. For ALK and ALKAL2 staining, BSA was replaced with Fetal Bovine Serum (FBS) at the same concentrations as above and then immunostained with either custom anti-ALKAL2 (rabbit, 1/1000, New England Peptide) or anti-phospho-ALK (rabbit, 1/100, New England Biolabs) overnight at 4° C. For secondary antibodies, we used either Alexa-488-conjugated anti-rabbit (1/2000, Invitrogen) or Alexa-555-conjugated anti-rabbit (1/2000, Invitrogen) for 1 hr at room temperature. Slides were washed in PBS twice and mounted with Aqua PolyMount (Polysciences Inc.) and imaged on a Zeiss 510 confocal microscope. Image analysis was conducted using ImageJ software as reported before (59).

For the neurite outgrowth assay, cultured DRG neurons were either treated with increasing concentrations (100 μM, 1 nM, 10 nM, 100 nM and 1 μM) of recombinant mouse ALKAL2 (MyBioSource LLC, MBS14253) or co-cultured with ALKAL2 expressing HEK cells (see above) in Neurobasal A medium. After 24 h, the cells were washed twice with HBSS, fixed in 4% PFA for 15 min., incubated in blocking solution (PBS+1% BSA) for 30 min. and then immunostained with anti-β-tubulin III antibody (rabbit, 1/1000, Sigma-Aldrich) overnight at 4° C. Cells were washed in PBS twice then incubated with a goat-anti rabbit IgG conjugated to Alexa Fluor 488 (1/2000, Invitrogen) for 1 h. After several washes, coverslips were mounted on slides and confocal images acquired. Alexa-488 antibody was visualized by excitation with an argon laser (514 nm) and emission detected using a long-pass 530 nm filter. Alexa-555 antibody was visualized by excitation at 543 nm with a HeNe laser, and emission detected using a 585-615 nm band-pass filter.

### Electrophysiological Measurements

Electrophysiological recordings were conducted using an external solution containing (in mM): 140.0 NaCl, 1.5 CaCl₂, 2.0 MgCl₂, 5.0 KCl, 10.0 HEPES, 10.0 D-glucose, pH 7.4 adjusted with NaOH, on the stage of an inverted epi-fluorescence microscope (Olympus IX51). DRG neuron were recorded based on size, knowing that TRPV1 is expressed in small neurons (<20 μM), and pHluorin fluorescent signal. Action potentials were recorded using current clamp. Borosilicate glass (Harvard Apparatus Ltd.) pipettes were pulled and polished to 2-5 MQ resistance with a DMZ-Universal Puller (Zeitz-Instruments GmbH.) and filled with an internal solution containing (in mM): 140.0 KCl, 5.00 NaCl, 1 CaCl₂, 1.0 EGTA, 10.0 HEPES, 1.0 MgCl₂, 3.0 ATP Na2, pH 7.3 adjusted with KOH. All solutions were prepared and used at room temperature (22±2° C.) and their osmolarity adjusted to 310 mOsm. For the current clamp experiments the spontaneous activity of the DRG neurons was recorded at room temperature (˜22° C.) for three minutes before application of ALKAL2 (1 μM applied to the bath at ˜1000 μm from the cell at a rate of 500 μl/min). Only the neurons in which the resting membrane potential was more negative than −40 mV and responded to capsaicin (1 μM) (Sigma-Aldrich) were used. Recordings were performed using an Axopatch 200B amplifier (Axon Instruments, Foster City, CA, USA). Current-clamp protocols were applied using pClamp 10.4 software (Axon Instruments). Data were filtered at 5 kHz (current clamp) and digitized at 10 kHz with a Digidata 1550 A converter (Axon Instruments). Average DRG neuron capacitance was 12.45±0.85 pF. Only the cells that exhibited a stable voltage control throughout the recording were used for analysis. For prolonged ALKAL2 treatment, neurons were incubated with a low concentration of 10 nM for 16 hours.

### Statistics

For electrophysiology, data analysis and offline leak subtraction were completed in Clampfit 10.4 (Axon Instruments). Statistical analysis and graphs were completed using Origin 7.0 analysis software (OriginLab, Northampton, MA, USA) or GraphPad Prism 7@ software (San Diego, CA, USA). Data are plotted as mean±SEM and numbers in parentheses reflect the number of cells (n). A paired t-test was used to compare data before and after drug treatment. One-way analysis of variance (ANOVA) followed by the Bonferroni post hoc test was used for multiple comparisons, with the criterion for statistical significance set at p<0.01. For behavior testing, statistical significance was evaluated by one-way or two-way ANOVA followed by Bonferroni test. Value of p s 0.05 was significant (*p≤0.05; **p≤0.01; ***p≤0.001, ****p<0.0001).

Human tissues were obtained under the approval of the French institution for organ transplantation (Agence de la Biomedecine, DC-2014-2420). All animal procedures were reviewed and approved by the University of Calgary Animal Care Committee and in accordance with the international guidelines for the ethical use of animals in research and guidelines of the Canadian Council on Animal Care. When possible behavioral testing was conducted in a double-blinded manner.

### REFERENCES AND NOTES

1. T. K. Lapointe, L. Basso, M. C. Iftinca, R. Flynn, K. Chapman, G. Dietrich, N. Vergnolle, C. Altier, TRPV1 sensitization mediates postinflammatory visceral pain following acute colitis, Am. J. Physiol.—Gastrointest. Liver Physiol. 309, G87-G99 (2015).
2. E. Bourinet, C. Altier, M. E. Hildebrand, T. Trang, M. W. Salter, G. W. Zamponi, Calcium-permeable ion channels in pain signaling, Physiol. Rev. 94, 81-140 (2014).
3. A. I. Basbaum, D. M. Bautista, G. Scherrer, D. Julius, Cellular and molecular mechanisms of pain., Cell 139, 267-84 (2009).
4. M. Tominaga, M. J. Caterina, A. B. Malmberg, T. A. Rosen, H. Gilbert, K. Skinner, B. E. Raumann, A. I. Basbaum, D. Julius, The Cloned Capsaicin Receptor Integrates Multiple Pain-Producing Stimuli, Neuron 21, 531-543 (1998).
5. J. C. Coleridge, H. M. Coleridge, Afferent vagal C fibre innervation of the lungs and airways and its functional significance., Rev. Physiol. Biochem. Pharmacol. 99, 1-110 (1984).
6. R. E. Miller, J. A. Block, A. M. Malfait, Nerve growth factor blockade for the management of osteoarthritis pain: What can we learn from clinical trials and preclinical models?, Curr. Opin. Rheumatol. 29, 110-118 (2017).
7. J. Collison, Anti-NGF therapy improves osteoarthritis pain, Nat. Rev. Rheumatol. 15, 450 (2019).
8. I. Drissi, W. A. Woods, C. G. Woods, Understanding the genetic basis of congenital insensitivity to pain, Br. Med. Bull. 133, 65-78 (2020).
9. L. J. Martin, S. B. Smith, A. Khoutorsky, C. A. Magnussen, A. Samoshkin, R. E. Sorge, C. Cho, N. Yosefpour, S. Sivaselvachandran, S. Tohyama, T. Cole, T. M. Khuong, E. Mir, D. G. Gibson, J. S. Wieskopf, S. G. Sotocinal, J. S. Austin, C. B. Meloto, J. H. Gitt, C. Gkogkas, N. Sonenberg, J. D. Greenspan, R. B. Fillingim, R. Ohrbach, G. D. Slade, C. Knott, R. Dubner, A. G. Nackley, A. Ribeiro-Da-Silva, G. G. Neely, W. Maixner, D. V. Zaykin, J. S. Mogil, L. Diatchenko, Epiregulin and EGFR interactions are involved in pain processing, J. Clin. Invest. 127, 3353-3366 (2017).
10. C. Rivat, C. Sar, I. Mechaly, J. P. Leyris, L. Diouloufet, C. Sonrier, Y. Philipson, O. Lucas, S. Mallie, A. Jouvenel, A. Tassou, H. Haton, S. Venteo, J. P. Pin, E. Trinquet, F. Charrier-Savournin, A. Mezghrani, W. Joly, J. Mion, M. Schmitt, A. Pattyn, F. Marmigere, P. Sokoloff, P. Carroll, D. Rognan, J. Valmier, Inhibition of neuronal FLT3 receptor tyrosine kinase alleviates peripheral neuropathic pain in mice, Nat. Commun. 9 (2018), doi:10.1038/s41467-018-03496-2.
11. H. Yuan, S. Du, L. Chen, X. Xu, Y. Wang, F. Ji, Hypomethylation of nerve growth factor (NGF) promotes binding of C/EBPa and contributes to inflammatory hyperalgesia in rats, J. Neuroinflammation 17 (2020), doi:10.1186/s12974-020-1711-1.
12. Y. Ben-Neriah, A. R. Bauskin, Leukocytes express a novel gene encoding a putative transmembrane protein-kinase devoid of an extracellular domain, Nature 333, 672-676 (1988).
13. A. Bernards, S. M. De la Monte, The Itk receptor tyrosine kinase is expressed in pre-B lymphocytes and cerebral neurons and uses a non-AUG translational initiator, EMBO J. 9, 2279-2287 (1990).
14. T. Iwahara, J. Fujimoto, D. Wen, R. Cupples, N. Bucay, T. Arakawa, S. Mori, B. Ratzkin, T. Yamamoto, Molecular characterization of ALK, a receptor tyrosine kinase expressed specifically in the nervous system, Oncogene 14, 439-449 (1997).
15. S. W. Morris, M. N. Kirstein, M. B. Valentine, K. G. Dittmer, D. N. Shapiro, D. L. Saltman, A. T. Look, Fusion of a kinase gene, ALK, to a nucleolar protein gene, NPM, in non-Hodgkin's lymphoma, Science (80-.). 263, 1281-1284 (1994).
16. B. Golding, A. Luu, R. Jones, A. M. Viloria-Petit, The function and therapeutic targeting of anaplastic lymphoma kinase (ALK) in non-small cell lung cancer (NSCLC), Mol. Cancer 17 (2018), doi:10.1186/s12943-018-0810-4.
17. R. M. Trigg, S. D. Turner, ALK in neuroblastoma: Biological and therapeutic implications, Cancers (Basel). 10 (2018), doi:10.3390/cancers10040113.
18. J. Y. Gouzi, C. Moog-Lutz, M. Vigny, N. Brunet-de Carvalho, Role of the subcellular localization of ALK tyrosine kinase domain in neuronal differentiation of PC12 cells, J. Cell Sci. 118, 5811-5823 (2005).
19. A. Motegi, J. Fujimoto, M. Kotani, H. Sakuraba, T. Yamamoto, ALK receptor tyrosine kinase promotes cell growth and neurite outgrowth, J. Cell Sci. 117, 3319-3329 (2004).
20. S. Yao, M. Cheng, Q. Zhang, M. Wasik, R. Kelsh, C. Winkler, Anaplastic Lymphoma Kinase Is Required for Neurogenesis in the Developing Central Nervous System of Zebrafish, PLoS One 8 (2013), doi:10.1371/journal.pone.0063757.
21. F. Facchinetti, L. Friboulet, Lorlatinib in ALK- and ROS1-positive NSCLC: The future has a start, Transl. Lung Cancer Res. 7, S103-S106 (2018).
22. R. Roskoski, Properties of FDA-approved small molecule protein kinase inhibitors: A 2020 update, Pharmacol. Res. 152 (2020), doi:10.1016/j.phrs.2019.104609.
23. P. Wu, T. E. Nielsen, M. H. Clausen, FDA-approved small-molecule kinase inhibitors, Trends Pharmacol. Sci. 36, 422-439 (2015).
24. H. Y. Zou, L. Friboulet, D. P. Kodack, L. D. Engstrom, Q. Li, M. West, R. W. Tang, H. Wang, K. Tsaparikos, J. Wang, S. Timofeevski, R. Katayama, D. M. Dinh, H. Lam, J. L. Lam, S. Yamazaki, W. Hu, B. Patel, D. Bezwada, R. L. Frias, E. Lifshits, S. Mahmood, J. F. Gainor, T. Affolter, P. B. Lappin, H. Gukasyan, N. Lee, S. Deng, R. K. Jain, T. W. Johnson, A. T. Shaw, V. R. Fantin, T. Smeal, PF-06463922, an ALK/ROS1 Inhibitor, Overcomes Resistance to First and Second Generation ALK Inhibitors in Preclinical Models, Cancer Cell 28, 70-81 (2015).
25. H. Huang, Anaplastic lymphoma kinase (Alk) receptor tyrosine kinase: A catalytic receptor with many faces, Int. J. Mol. Sci. 19 (2018), doi:10.3390/ijms19113448.
26. H. Zhang, L. I. Pao, A. Zhou, A. D. Brace, R. Halenbeck, A. W. Hsu, T. L. Bray, K. Hestir, E. Bosch, E. Lee, G. Wang, H. Liu, B. R. Wong, W. M. Kavanaugh, L. T. Williams, Deorphanization of the human leukocyte tyrosine kinase (LTK) receptor by a signaling screen of the extracellular proteome, Proc. Natl. Acad. Sci. U.S.A 111, 15741-15745 (2014).
27. J. Guan, G. Umapathy, Y. Yamazaki, G. Wolfstetter, P. Mendoza, K. Pfeifer, A. Mohammed, F. Hugosson, H. Zhang, A. W. Hsu, R. Halenbeck, B. Hallberg, R. H. Palmer, FAM150A and FAM150B are activating ligands for anaplastic lymphoma kinase, Elife 4 (2015), doi:10.7554/eLife.09811.
28. A. V. Reshetnyak, P. B. Murray, X. Shi, E. S. Mo, J. Mohanty, F. Tome, H. Bai, M. Gunel, I. Lax, J. Schlessinger, Augmentor a and p (FAM150) are ligands of the receptor tyrosine kinases ALK and LTK: Hierarchy and specificity of ligand-receptor interactions, Proc. Natl. Acad. Sci. U.S.A 112, 15862-15867 (2015).
29. D. J. Cavanaugh, A. T. Chesler, J. M. Braz, N. M. Shah, D. Julius, A. I. Basbaum, Restriction of Transient Receptor Potential Vanilloid-1 to the Peptidergic Subset of Primary Afferent Neurons Follows Its Developmental Downregulation in Nonpeptidergic Neurons, J. Neurosci. 31, 10119-10127 (2011).
30. G. Miesenböck, D. A. De Angelis, J. E. Rothman, Visualizing secretion and synaptic transmission with pH-sensitive green fluorescent proteins, Nature 394, 192-195 (1998).
31. M. Iftinca, R. Flynn, L. Basso, H. Melo, R. Aboushousha, L. Taylor, C. Altier, The stress protein heat shock cognate 70 (Hsc70) inhibits the Transient Receptor Potential Vanilloid type 1 (TRPV1) channel, Mol. Pain 12 (2016), doi:10.1177/1744806916663945.
32. Q. Tian, J. Hu, C. Xie, K. Mei, C. Pham, X. Mo, R. Hepp, S. Soares, F. Nothias, Y. Wang, Q. Liu, F. Cai, B. Zhong, D. Li, J. Yao, Recovery from tachyphylaxis of TRPV1 coincides with recycling to the surface membrane, Proc. Natl. Acad. Sci. U.S.A 116, 5170-5175 (2019).
33. S. Hunskaar, O. B. Fasmer, K. Hole, Formalin test in mice, a useful technique for evaluating mild analgesics, J. Neurosci. Methods 14, 69-76 (1985).
34. N. Sharma, K. Flaherty, K. Lezgiyeva, D. E. Wagner, A. M. Klein, D. D. Ginty, The emergence of transcriptional identity in somatosensory neurons, Nature 577, 392-398 (2020).
35. W. Renthal, I. Tochitsky, L. Yang, Y. C. Cheng, E. Li, R. Kawaguchi, D. H. Geschwind, C. J. Woolf, Transcriptional Reprogramming of Distinct Peripheral Sensory Neuron Subtypes after Axonal Injury, Neuron 108, 128-144.e9 (2020).
36. A. Zeisel, H. Hochgerner, P. Lönnerberg, A. Johnsson, F. Memic, J. van der Zwan, M. Haring, E. Braun, L. E. Borm, G. La Manno, S. Codeluppi, A. Furlan, K. Lee, N. Skene, K. D. Harris, J. Hjerling-Leffler, E. Arenas, P. Ernfors, U. Marklund, S. Linnarsson, Molecular Architecture of the Mouse Nervous System, Cell 174, 999-1014.e22 (2018).
37. A. Fadeev, P. Mendoza-Garcia, U. Irion, J. Guan, K. Pfeifer, S. Wiessner, F. Serluca, A. P. Singh, C. Nusslein-Volhard, R. H. Palmer, ALKALs are in vivo ligands for ALK family receptor tyrosine kinases in the neural crest and derived cells, Proc. Natl. Acad. Sci. U.S.A 115, E630-E638 (2018).
38. J. Degoutin, N. Brunet-De Carvalho, C. Cifuentes-Diaz, M. Vigny, ALK (Anaplastic Lymphoma Kinase) expression in DRG neurons and its involvement in neuron-Schwann cells interaction, Eur. J. Neurosci. 29, 275-286 (2009).
39. B. Duan, L. Cheng, S. Bourane, O. Britz, C. Padilla, L. Garcia-Campmany, M. Krashes, W. Knowlton, T. Velasquez, X. Ren, S. E. Ross, B. B. Lowell, Y. Wang, M. Goulding, Q. Ma, Identification of spinal circuits transmitting and gating mechanical pain, Cell 159, 1417-1432 (2014).
40. V. Verma, S. Khoury, M. Parisien, C. Cho, W. Maixner, L. J. Martin, L. Diatchenko, The dichotomous role of epiregulin in pain, Pain 161, 1052-1064 (2020).
41. D. J. Cavanaugh, A. T. Chesler, A. C. Jackson, Y. M. Sigal, H. Yamanaka, R. Grant, D. O'Donnell, R. A. Nicoll, N. M. Shah, D. Julius, A. I. Basbaum, Trpv1 reporter mice reveal highly restricted brain distribution and functional expression in arteriolar smooth muscle cells, J. Neurosci. 31, 5067-5077 (2011).
42. C. R. Donnelly, O. Chen, R. R. Ji, How Do Sensory Neurons Sense Danger Signals?, Trends Neurosci. 43, 822-838 (2020).
43. M. J. Lacagnina, L. R. Watkins, P. M. Grace, Toll-like receptors and their role in persistent pain, Pharmacol. Ther. 184, 145-158 (2018).
44. Y. Shinoda, S. Tatsumi, E. Yoshida, T. Takahashi, K. Eto, T. Kaji, Y. Fujiwara, Gene expression profiles in the dorsal root ganglia of methylmercury-exposed rats, J. Toxicol. Sci. 44, 549-558 (2019).
45. E. S. Mo, Q. Cheng, A. V. Reshetnyak, J. Schlessinger, S. Nicoli, Alk and Ltk ligands are essential for iridophore development in zebrafish mediated by the receptor tyrosine kinase Ltk, Proc. Natl. Acad. Sci. U.S.A 114, 12027-12032 (2017).
46. G. E. Stoica, A. Kuo, A. Aigner, I. Sunitha, B. Souttou, C. Malerczyk, D. J. Caughey, D. Wen, A. Karavanov, A. T. Riegel, A. Wellstein, Identification of Anaplastic Lymphoma Kinase as a Receptor for the Growth Factor Pleiotrophin, J. Biol. Chem. 276, 16772-16779 (2001).
47. G. E. Stoica, A. Kuo, C. Powers, E. T. Bowden, E. B. Sale, A. T. Riegel, A. Wellstein, Midkine binds to anaplastic lymphoma kinase (ALK) and acts as a growth factor for different cell types, J. Biol. Chem. 277, 35990-35998 (2002).
48. B. Hallberg, R. H. Palmer, The role of the ALK receptor in cancer biology, Ann. Oncol. 27, iii4-iii15 (2016).
49. M. Orthofer, A. Valsesia, R. Magi, Q. P. Wang, J. Kaczanowska, I. Kozieradzki, A. Leopoldi, D. Cikes, L. M. Zopf, E. O. Tretiakov, E. Demetz, R. Hilbe, A. Boehm, M. Ticevic, M. Nõukas, A. Jais, K. Spirk, T. Clark, S. Amann, M. Lepamets, C. Neumayr, C. Arnold, Z. Dou, V. Kuhn, M. Novatchkova, S. J. F. Cronin, U. J. F. Tietge, S. Müller, J. A. Pospisilik, V. Nagy, C. C. Hui, J. Lazovic, H. Esterbauer, A. Hagelkruys, I. Tancevski, F. W. Kiefer, T. Harkany, W. Haubensak, G. G. Neely, A. Metspalu, J. Hager, N. Gheldof, J. M. Penninger, Identification of ALK in Thinness, Cell 181, 1246-1262.e22 (2020).
50. G. F. Gibbs, P. D. Drummond, P. M. Finch, J. K. Phillips, Unravelling the pathophysiology of complex regional pain syndrome: Focus on sympathetically maintained pain, Clin. Exp. Pharmacol. Physiol. 35, 717-724 (2008).
51. M. Khan, J. Lin, G. Liao, Y. Tian, Y. Liang, R. Li, M. Liu, Y. Yuan, ALK inhibitors in the treatment of ALK positive NSCLC, Front. Oncol. 9 (2019), doi:10.3389/fonc.2018.00557.
52. A. T. Shaw, T. M. Kim, L. Crinò, C. Gridelli, K. Kiura, G. Liu, S. Novello, A. Bearz, O. Gautschi, T. Mok, M. Nishio, G. Scagliotti, D. R. Spigel, S. Deudon, C. Zheng, S. Pantano, P. Urban, C. Massacesi, K. Viraswami-Appanna, E. Felip, Ceritinib versus chemotherapy in patients with ALK-rearranged non-small-cell lung cancer previously given chemotherapy and crizotinib (ASCEND-5): a randomised, controlled, open-label, phase 3 trial, Lancet Oncol. 18, 874-886 (2017).
53. J. C. Soria, D. S. W. Tan, R. Chiari, Y. L. Wu, L. Paz-Ares, J. Wolf, S. L. Geater, S. Orlov, D. Cortinovis, C. J. Yu, M. Hochmair, A. B. Cortot, C. M. Tsai, D. Moro-Sibilot, R. G. Campelo, T. McCulloch, P. Sen, M. Dugan, S. Pantano, F. Branle, C. Massacesi, G. de Castro, First-line ceritinib versus platinum-based chemotherapy in advanced ALK-rearranged non-small-cell lung cancer (ASCEND-4): a randomised, open-label, phase 3 study, Lancet 389, 917-929 (2017).
54. C. Dromard, H. Guillon, V. Rigau, C. Ripoll, J. C. Sabourin, F. E. Perrin, F. Scamps, S. Bozza, P. Sabatier, N. Lonjon, H. Duffau, F. Vachiery-Lahaye, M. Prieto, C. Tran Van Ba, L. Deleyrolle, A. Boularan, K. Langley, M. Gaviria, A. Privat, J. P. Hugnot, L. Bauchet, Adult human spinal cord harbors neural precursor cells that generate neurons and glial cells in vitro, J. Neurosci. Res. 86, 1916-1926 (2008).
55. V. M. Gadotti, H. You, R. R. Petrov, N. D. Berger, P. Diaz, G. W. Zamponi, Analgesic effect of a mixed T-type channel inhibitor/CB2 receptor agonist, Mol. Pain 9 (2013), doi:10.1186/1744-8069-9-32.
56. J. Ferreira, M. M. Campos, J. B. Pesquero, R. C. AraUjo, M. Bader, J. B. Calixto, Evidence for the participation of kinins in Freund's adjuvant-induced inflammatory and nociceptive responses in kinin B1 and B2 receptor knockout mice, Neuropharmacology 41, 1006-1012 (2001).
57. A. B. Malmberg, A. I. Basbaum, Partial sciatic nerve injury in the mouse as a model of neuropathic pain: Behavioral and neuroanatomical correlates, Pain 76, 215-222 (1998).
58. V. M. Gadotti, A. G. Caballero, N. D. Berger, C. M. Gladding, L. Chen, T. A. Pfeifer, G. W. Zamponi, Small Organic Molecule Disruptors of Cav3.2—USP5 Interactions Reverse Inflammatory and Neuropathic Pain, Mol. Pain 11 (2015), doi:10.1186/s12990-015-0011-8.
59. C. Altier, A. Garcia-Caballero, B. Simms, H. You, L. Chen, J. Walcher, H. W. Tedford, T. Hermosilla, G. W. Zamponi, The Cavβ subunit prevents RFP2-mediated ubiquitination and proteasomal degradation of L-type channels, Nat. Neurosci. 14, 173-182 (2011).
60. A. Zeisel, H. Hochgerner, P. Lönnerberg, A. Johnsson, F. Memic, J. van der Zwan, M. Haring, E. Braun, L. E. Borm, G. La Manno, S. Codeluppi, A. Furlan, K. Lee, N. Skene, K. D. Harris, J. Hjerling-Leffler, E. Arenas, P. Ernfors, U. Marklund, S. Linnarsson, Molecular Architecture of the Mouse Nervous System, Cell 174, 999-1014.e22 (2018).

### Example 2

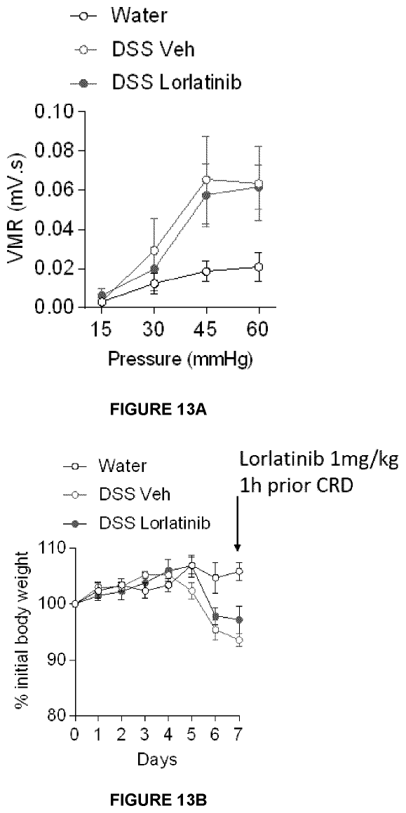

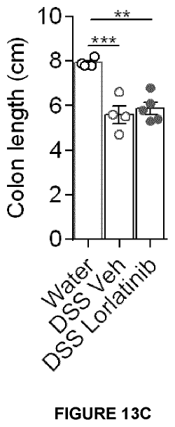

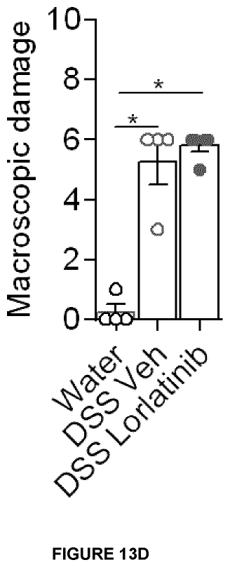

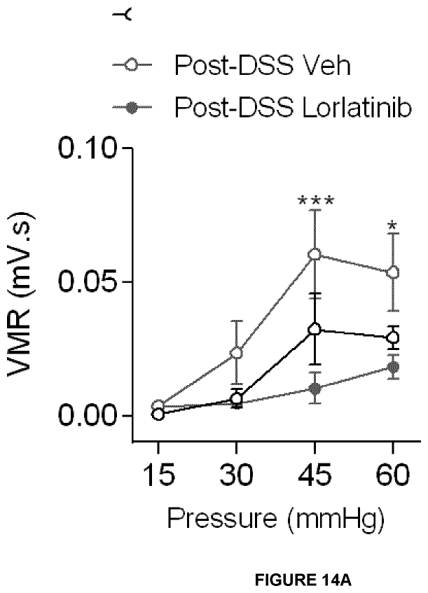

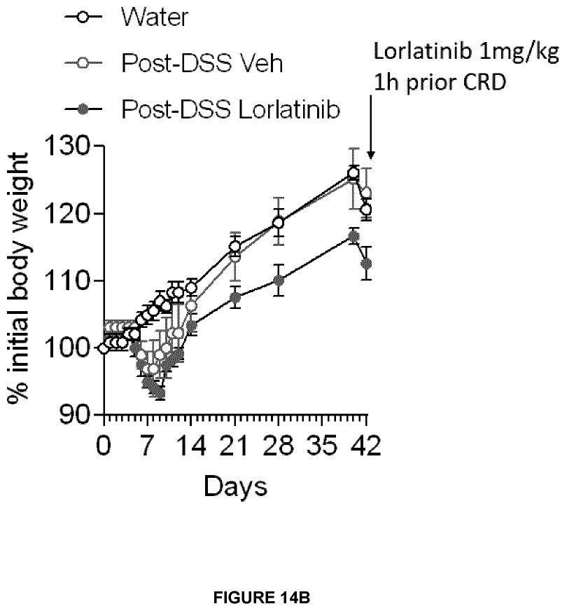

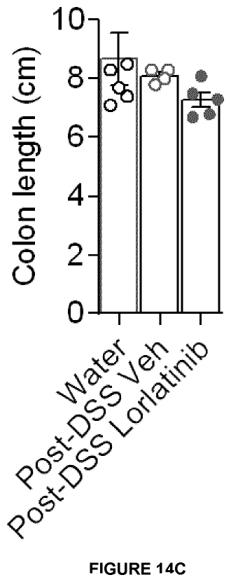

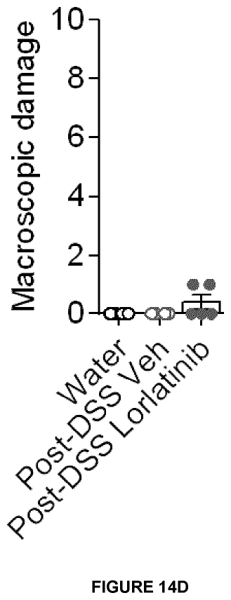

Chronic abdominal pain is a common symptom of inflammatory bowel diseases (IBD). We have developed a model of dextran sulfate sodium (DSS)-induced colitis that exhibits chronic visceral hypersensitivity (VHS) in the recovery phase of colitis (post-inflammatory colitis. Lapointe et al AJPGI. PMID: 26021808). We tested the effect of Lorlatinib on acute and chronic VHS (FIG. 13A-D and FIG. 14A-D).

### Results

Mice that received DSS developed intestinal inflammation characterized by a slight decrease in the body weight (FIG. 13A), decrease in colon length and increase in macroscopic damages (FIGS. 13B and C). As previously reported, colitis induces an increase in visceral hypersensitivity measured by VMR to colorectal distension. Inhibition of ALK with Lorlatinib does not affect VHS (FIG. 13D). We next evaluated the effect of Lorlatinib on post-colitis visceral hypersensitivity. As previously reported, post-colitis mice exhibited a significant increase in VMR to colorectal distension (FIG. 14D), despite complete resolution of inflammation (FIG. 14A-C). In contrast to the acute DSS model, inhibition of ALK with Lorlatinib normalized VHS in post-colitis mice (FIG. 14D). These results indicate that activation of ALK plays a central role in persistent VHS post colitis. ALK inhibition could be used as a treatment for IBD patients who experience chronic visceral pain during disease remission.

### Methodology

### Animals

We used adult male C57BL/6 J mice (6 to 8 weeks old) purchased from the Jackson Laboratory (#664). All experiments were conducted under protocols approved by the University of Calgary Animal Care Committee and in accordance with the international guidelines for the ethical use of animals in research and guidelines of the Canadian Council on Animal Care. Animals were housed at a maximum of three per cage (30×20×15 cm), with water and food ad libitum. They were kept in controlled temperature of 23±1° C. on a 12 h light/dark cycles (lights on at 7:00 a.m.). Different cohorts of mice were used for each test.

### Induction of Colitis and Inflammation Assessment.

Acute colonic inflammation was induced by administration of 2.5% (wt/vol) Dextran Sodium Sulfate (DSS) (Alfa Aesar, Cat. No. J63606) in drinking water for 7 days. Visceral hypersensitivity post-colitis was induced as previously described (PMID: 26021808). Briefly, colitis was induced by administration of 2.5% DSS (wt/vol) in drinking water for 5 days. On day 5, DSS was removed and replaced by water to allow mice to recover for 5 weeks. Body weight was monitored daily during DSS exposure and weekly during the recovery period. Macroscopic damage of the colon was assessed and scored based on the following parameters: adhesions (0, absent; 1, moderate; 2, severe); edema (0, absent; 1, moderate; 2, severe); strictures (0, absent; 1, 1; 2, 2; 3, >2); blood (0, absent; 1, present); ulcer (0, absent; 1, present); and mucus (0, absent; 1, present).

### Visceromotor Response to Colorectal Distension.

VMR to colorectal distension (CRD) was performed as previously described (PMID: 26021808). Briefly, mice were anesthetized with xylazine/ketamine and implanted with two electrodes in the abdominal external oblique muscle. The mice were allowed to recover for 2 days before visceral sensitivity assessment. For recording, electrodes were connected to an electromyogram acquisition system via a Bio Amplifier (both from ADlnstruments), and a 10.5 mm diameter balloon catheter (Edwards Life-Sciences, Cat. No. 12TLW404F) was inserted 5 mm proximal to the mouse rectum. Mice were subjected to four 10-s distensions (15, 30, 45, and 60 mmHg pressure) with 5-min rest intervals. Electromyographic activity of the abdominal muscles was recorded, and VMR was calculated using LabChart 7 (ADlnstruments). Mice received Lorlatinib (1 mg/Kg) 1 hour before measuring visceromotor responses (VMR) to colorectal distension.

### Statistical Analyses.

Statistical analyses were performed with GraphPad Prism 7® software. Normal distribution was verified using D'Agostino-Pearson normality Test. For Gaussian data, Student's t-test was used to assess statistical significance when comparing two means, One-Way ANOVA followed by the Tukey post hoc test was used to compare more than two groups and Two-way ANOVA followed by Bonferroni (two groups) and Tukey post hoc test (for more than two groups) for multiple comparisons. For non-Gaussian data, the non-parametric Mann Whitney U test was used to assess statistical significance when comparing two means, Kruskal-Wallis followed by the Dunn's post hoc test was used to compare more than two groups Statistical significance was established at P s 0.05. Values were expressed as means±standard error mean (SEM).

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of treating pain in a subject in need thereof comprising administering to the subject an anaplastic lymphoma kinase (ALK) inhibitor.

2. The method of claim 1, wherein the ALK inhibitor is a small molecule, an antibody, a polynucleotide, or a pharmaceutical composition thereof.

3. The method of claim 1, wherein the ALK inhibitor is Crizotinib, Alectinib, Brigatinib, Lorlatinib, or TPX-0131.

4. The method of claim 2, wherein the antibody is a monoclonal antibody, preferably mAb30, mAb49, or anti-Human ALK/CD246 Monoclonal Antibody.

5. The method of claim 2, wherein the polynucleotide is an antisense polynucleotide to target human ALK hASO-1: 5′-GAAGCAGAGCGCACACAAAA (SEQ ID NO: 25), hASO-2: 5′-TCCTCATCCATGGGCTCAGA (SEQ ID NO: 26), hASO-3: 5′-CGCTGAGGTTGAACTGGAGT (SEQ ID NO: 27).

6. The method of claim 1, wherein the pain is inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer, including “break-through pain” and pain associated with terminal cancer, peripheral neuropathy, post-herpetic neuralgia, or pain from colitis.

7. The method of claim 1, wherein the subject is a cat, a dog, a cow, a horse, a pig, a sheep, a goat, a mouse, a rabbit, a rat, a guinea pig, a non-human mammal, a non-human primate, a rodent, a bird, a reptile, an amphibian, a fish, or a human.

8. A method of treating pain in a subject in need thereof comprising administering to the subject an ALKAL2 inhibitor.

9. The method of claim 8, wherein the ALKAL2 inhibitor is a small molecule, an antibody, a polynucleotide, or a pharmaceutical composition thereof.

10. The method of claim 9, wherein the polynucleotide is an antisense polynucleotide Human specific ASO: Design #1: hASO-1 DNA Sequence: 5′-CAAGTACACTGATTTATCGA (SEQ ID NO: 4). Design #2: hASO-2 DNA Sequence: 5′-GCACTACACGTCAAATGTGG (SEQ ID NO: 5). Design #3: hASO-3 DNA Sequence: 5′-ACAATAGCTGGAATACTATT (SEQ ID NO: 6).

11. The method of claim 9, wherein the polynucleotide in an antisense polynucleotide Design #1: ASO-1 DNA Sequence: 5′-AAGTGCTTGCTGCACTTCGG (SEQ ID NO: 1), Design #2: ASO-2 DNA Sequence: 5′-GATGGTGCAGTCTCTCGTGT (SEQ ID NO: 2), or Design #3: ASO-3 DNA Sequence: 5′-TGGTGTGTCGCTCCTTTGCA (SEQ ID NO: 3).

12. The method of claim 8, wherein the pain is inflammatory pain, post-operative incision pain, neuropathic pain, fracture pain, osteoporotic fracture pain, post-herpetic neuralgia, cancer pain, pain resulting from burns, pain associated with burn or wound, pain associated with trauma (including traumatic head injury), pain associated with musculoskeletal disorders such as rheumatoid arthritis, osteoarthritis, ankylosing spondylitis, seronegative (non-rheumatoid) arthropathies, non-articular rheumatism and periarticular disorders, and pain associated with cancer, including “break-through pain” and pain associated with terminal cancer, peripheral neuropathy, post-herpetic neuralgia or pain from colitis.

13. The method of claim 8, wherein the subject is a cat, a dog, a cow, a horse, a pig, a sheep, a goat, a mouse, a rabbit, a rat, a guinea pig, a non-human mammal, a non-human primate, a rodent, a bird, a reptile, an amphibian, a fish, or a human.

14. The method of claim 8, wherein administration is topical, intradermal, intramuscular, intraperitoneal, intravenous, subcutaneous, intranasal, epidural, or oral routes.

15.-30. (canceled)

31. The method of claim 1, wherein administration is topical, intradermal, intramuscular, intraperitoneal, intravenous, subcutaneous, intranasal, epidural, or oral routes.


SEQUENCE LISTING (27 sequences)

SEQ ID NO: 1 - DNA, 20 bp, synthetic construct
  source 1..20: mol_type = other DNA
aagtgcttgctgcacttcgg

SEQ ID NO: 2 - DNA, 20 bp, synthetic construct
  source 1..20: mol_type = other DNA
gatggtgcagtctctcgtgt

SEQ ID NO: 3 - DNA, 20 bp, synthetic construct
  source 1..20: mol_type = other DNA
tggtgtgtcgctcctttgca

SEQ ID NO: 4 - DNA, 20 bp, synthetic construct
  source 1..20: mol_type = other DNA
caagtacactgatttatcga

SEQ ID NO: 5 - DNA, 20 bp, synthetic construct
  source 1..20: mol_type = other DNA
gcactacacgtcaaatgtgg

SEQ ID NO: 6 - DNA, 20 bp, synthetic construct
  source 1..20: mol_type = other DNA
acaatagctggaatactatt

SEQ ID NO: 7 - DNA, 21 bp, synthetic construct
  source 1..21: mol_type = other DNA
tgaggttgctagttgagctgg

SEQ ID NO: 8 - DNA, 22 bp, synthetic construct
  source 1..22: mol_type = other DNA
aggaacaatctccactctctgc

SEQ ID NO: 9 - DNA, 18 bp, synthetic construct
  source 1..18: mol_type = other DNA
ccatggagaaggctgggg

SEQ ID NO: 10 - DNA, 20 bp, synthetic construct
  source 1..20: mol_type = other DNA
caaagttgtcatggatgacc

SEQ ID NO: 11 - DNA, 21 bp, synthetic construct
  source 1..21: mol_type = other DNA
caacaagaaggggcttacacc

SEQ ID NO: 12 - DNA, 22 bp, synthetic construct
  source 1..22: mol_type = other DNA
tctggagaatgtaggccaagac

SEQ ID NO: 13 - DNA, 18 bp, synthetic construct
  source 1..18: mol_type = other DNA
cccaagggttcagaagag

SEQ ID NO: 14 - DNA, 19 bp, synthetic construct
  source 1..19: mol_type = other DNA
gggcatcctcgatgagact

SEQ ID NO: 15 - DNA, 19 bp, synthetic construct
  source 1..19: mol_type = other DNA
gagccacagcctgtgccct

SEQ ID NO: 16 - DNA, 21 bp, synthetic construct
  source 1..21: mol_type = other DNA
cgtgctagtggctaaggcatc

SEQ ID NO: 17 - DNA, 20 bp, synthetic construct
  source 1..20: mol_type = other DNA
aaagcaggagctggggacta

SEQ ID NO: 18 - DNA, 20 bp, synthetic construct
  source 1..20: mol_type = other DNA
tgttcgattttgcggtcaga

SEQ ID NO: 19 - DNA, 19 bp, synthetic construct
  source 1..19: mol_type = other DNA
gtcttaggcgccttcgtgg

SEQ ID NO: 20 - DNA, 20 bp, synthetic construct
  source 1..20: mol_type = other DNA
ttgcacgttacactgcttgc

SEQ ID NO: 21 - DNA, 20 bp, synthetic construct
  source 1..20: mol_type = other DNA
ggtgctgctggaaacacttc

SEQ ID NO: 22 - DNA, 21 bp, synthetic construct
  source 1..21: mol_type = other DNA
tggagttcattcctgctcctg

SEQ ID NO: 23 - DNA, 19 bp, synthetic construct
  source 1..19: mol_type = other DNA
cagatccccgacacttgtg

SEQ ID NO: 24 - DNA, 20 bp, synthetic construct
  source 1..20: mol_type = other DNA
tgtgctgcttgtactggcct

SEQ ID NO: 25 - DNA, 20 bp, synthetic construct
  source 1..20: mol_type = other DNA
gaagcagagcgcacacaaaa

SEQ ID NO: 26 - DNA, 20 bp, synthetic construct
  source 1..20: mol_type = other DNA
tcctcatccatgggctcaga

SEQ ID NO: 27 - DNA, 20 bp, synthetic construct
  source 1..20: mol_type = other DNA
cgctgaggttgaactggagt